(19) United States
(12) Patent Application Publication
(10) Pub. No.: US 2026/0108887 A1
(43) Pub. Date: Apr. 23, 2026

(54) MICROFLUIDIC BEAD TRAPPING DEVICES AND METHODS FOR NEXT GENERATION SEQUENCING LIBRARY PREPARATION

(71) Applicant: Roche Sequencing Solutions, Inc., Pleasanton, CA (US)

(72) Inventors: Yann Astier, Livermore, CA (US); Daniel Burgess, Black Earth, WI (US); Ulrich Schlecht, Sunnyvale, CA (US); Jaeyoung Yang, Pleasanton, CA (US)

(21) Appl. No.: 19/280,538

(22) Filed: Jul. 25, 2025

Related U.S. Application Data
(62) Division of application No. 17/759,133, now abandoned.
(60) Provisional application No. 62/964,388, filed on Jan. 22, 2020.

(51) Int. Cl. B01L 3/00; C12N 15/10

(52) U.S. Cl. CPC B01L 3/502761; C12N 15/1006; B01L 2200/0668; B01L 2300/0877; B01L 2300/0883

(57) ABSTRACT

The present disclosure is directed to automated systems including a microfluidic chip having one or more independently operable processing conduits. In some embodiments, the automated systems are suitable for use in sample cleanup and/or target enrichment processes, such as sample cleanup and/or target enrichment processes conducted prior to sequencing.

### BACKGROUND OF THE DISCLOSURE

Microfluidic systems are of significant value for acquiring and analyzing chemical and biological information using very small volumes of liquid. Microfluidics can be broadly defined as systems leveraging micrometer scale channels to manipulate and process low volume fluid samples. Use of microfluidic systems can increase the response time of reactions, minimize sample volume, and lower reagent and consumables consumption. Performing reactions in microfluidic volumes also enhances safety and reduces disposal quantities when volatile or hazardous materials are used or generated. Such microfluidic devices are used, for example, in medical diagnostics, genomic analysis, DNA forensics, and “lab-on-a-chip” chemical analyzers; and they can be fabricated using common microfabrication techniques, such as photolithography.

Microfluidic particle separation involves the capture, isolation, and collection of target particles from impure or complex samples and is widely used in sorting, purification, enrichment, and detection of cells in cell biology, drug discovery, and clinical diagnostics. A number of methods currently exist for particle separation on microfluidic platforms, including magnetic-activated separation. For example, methods based on magnetic control utilize surface-functionalized magnetic beads to capture target particles through specific binding and then to separate the target particles by magnetic manipulation. This separation scheme relies on the interaction of chemical bonds rather than geometrical or physical properties of the particles and hence allows highly specific and selective particle separation.

There are, in general, two operating modes for magnetically based microfluidic particle separation, i.e., batch mode and continuous flow mode. In the batch mode, target-bound magnetic beads are retained on a solid surface and subsequently released, following the removal of nontarget particles with a liquid phase. Magnetic bead beds and sifts have, for example, been developed for this purpose but have limited separation efficiency. A number of devices have attempted to address this issue with various magnet designs, including a quadruple electromagnet, a planar electromagnet, nickel posts, etc. In addition, planar electromagnets can be integrated on chip with microvalves and micropumps to enable fully automated functionalities, such as fluid actuation and particle mixing. Unfortunately, batch-mode designs suffer from several inherent limitations, including prolonged durations of operation, complicated fluidic handling, and, most importantly, significant contamination due to nonspecific trapping of impurities that are sequestered in the beads.

### BRIEF SUMMARY OF THE DISCLOSURE

The present disclosure is directed to a microfluidic device including one or more independently operable processing conduits for purifying target molecules, such as nucleic acids, within an input sample where the processing conduits do not include any mechanically moving components. Moreover, the microfluidic devices and processing conduits of the present disclosure do not rely on magnetic separation techniques. Additionally, the microfluidic devices of the present disclosure have reduced complexity as compared with those microfluidic devices employing mechanically moving parts. Moreover, the microfluidic devices of the present disclosure employ a closed system which mitigates sample contamination. Further, given the design of the independently operable processing conduits of the presently disclosed microfluidic devices, bead loss, and hence target molecule loss, is advantageously mitigated. These and other advantages are described herein.

A first aspect of the present disclosure is a microfluidic chip including a processing conduit having a chamber including a plurality of beads, wherein a first portion of a wall of the chamber includes a first aperture in fluidic communication with an inlet channel, a second portion of the wall of the chamber includes a second aperture in fluidic communication with an outlet channel, and a third portion of the wall of the chamber comprises a ductal opening in fluidic communication with a duct; wherein the first and second apertures are smaller than an average diameter of the plurality of beads within the chamber, and wherein the ductal opening is larger than the average diameters of the plurality of beads within the chamber. In some embodiments, the microfluidic chip comprises no mechanically moving parts. In some embodiments, the microfluidic chip is comprised of a non-magnetic material. In some embodiments, the plurality of beads are non-magnetic beads.

In some embodiments, the chamber comprises a volume ranging from between about 0.1 μL to about 5 mL. In some embodiments, the volume ranges from between about 0.1 mL to about 1 mL. In some embodiments, the chamber comprises between 1 post and about 1,000,000 posts. In some embodiments, the posts extend from either a ceiling or a floor of the chamber. In some embodiments, the posts bridge a ceiling and a floor of the chamber. In some embodiments, at least the inlet channel comprises between 1 and about 1,000,000 posts.

In some embodiments, the microfluidic chip comprises one processing conduit. In some embodiments, the microfluidic chip comprises between 2 and 50 independently operable processing conduits. In some embodiments, the microfluidic chip comprises between 2 and 20 independently operable processing conduits.

A second aspect of the present disclosure is a microfluidic chip including a processing conduit having two or more chambers, wherein any two adjacent chambers of the two or more chambers are fluidically coupled to one another through a transfer channel, and wherein at least one of the two or more chambers includes a plurality of beads; wherein a portion of a wall of a first of the two or more chambers includes a first aperture in fluidic communication with an inlet channel; a portion of a wall of a second of the two or more chambers comprises a second aperture in fluidic communication with an outlet channel; and wherein at least one of the two or more chambers comprises a ductal opening in fluidic communication with a duct; wherein the first and second apertures are smaller than an average diameter of the plurality of beads within the at least one of the two or more chambers, and wherein the ductal opening is larger than the average diameters of the plurality beads within the at least one of the two or more chambers. In some embodiments, the microfluidic chip comprises no mechanically moving parts. In some embodiments, the microfluidic chip is comprised of a non-magnetic material. In some embodiments, the plurality of beads are non-magnetic beads.

In some embodiments, the transfer conduit comprises a serpentine shape. In some embodiments, the transfer channel has a cross-sectional height and width which is greater than the average diameter of the plurality of beads. In some embodiments, the plurality of beads are flowable through the transfer conduit.

In some embodiments, the processing conduit includes two chambers. In some embodiments, the two chambers are fluidically coupled to each other through a serpentine channel. In some embodiments, the plurality of beads are flowable from the first chamber to the second chamber through the serpentine transfer channel.

In some embodiments, the processing conduit includes three chambers. In some embodiments, a first of the three chambers is fluidically coupled to the inlet channel, a middle (second) of the three chambers is fluidically coupled to each of the first and third chambers through two transfer channels, and the third chamber is fluidically coupled to the outlet channel.

In some embodiments, the two or more chambers each comprise between 1 and about 1,000,000 posts. In some embodiments, the posts extend from either a ceiling or a floor of the chamber. In some embodiments, at least the inlet channel comprises between 1 and about 1,000,000 posts. In some embodiments, the two or more chambers each comprise a volume ranging from between about 0.1 μL to about 5 mL. In some embodiments, volume ranges from between about 0.1 mL to about 1 mL.

In some embodiments, the microfluidic chip comprises one processing conduit. In some embodiments, the microfluidic chip comprises between 2 and 50 independently operable processing conduits. In some embodiments, the microfluidic chip comprises between 2 and 20 independently operable processing conduits.

A third aspect of the present disclosure is a method of obtaining a population of target nucleic acid sequences for sequencing comprising: (a) fragmenting an obtained genomic sample to provide a population of nucleic acid fragments; (b) introducing a pool of oligonucleotide probes to the population of nucleic acid fragments to form target-probe complexes, wherein the pool of oligonucleotide probes comprise reference nucleic acid sequences capable of hybridizing to complementary nucleic acid sequences within the population of nucleic acid fragments and wherein the oligonucleotide probes comprise a first member of a pair of specific binding entities, (c) flowing a solution including the formed target-probe complexes through a processing conduit of a microfluidic chip, wherein the processing conduit comprises a chamber including a plurality of beads, wherein the plurality of beads are functionalized with a second member of the pair of specific binding entities; (d) flowing at least one buffer through the processing conduit to remove off-target fragments; and (e) flowing at least one reagent through the processing conduit to obtain the target nucleic acid sequences. In some embodiments, the first moiety is biotin. In some embodiments, the second moiety is streptavidin.

In some embodiments, the flowing of the at least one buffer is sequentially repeated at least twice. In some embodiments, the flowing of the at least one buffer is sequentially repeated at least three times. In some embodiments, each sequentially flowed buffer is the same. In some embodiments, each sequentially flowed buffer is different.

In some embodiments, the at least one reagent is a buffer having a temperature ranging from between about 80° C. to about 105° C. In some embodiments, the at least one reagent is a buffer, and wherein the processing conduit is heated to a temperature ranging from between about 90° C. to about 100° C. In some embodiments, the reagent is an enzyme.

In some embodiments, the method further comprises ligating adaptors to the population of nucleic acid fragments after fragmentation of the obtained genomic sample. In some embodiments, the method further comprises sequencing the population of target nucleic acid sequences.

In some embodiments, the plurality of beads are non-magnetic beads. In some embodiments, the microfluidic chip comprises no mechanically moving parts. In some embodiments, the microfluidic chip is comprised of a non-magnetic material.

In a fourth aspect of the present disclosure is a method of obtaining a population of target nucleic acid sequences for sequencing comprising: (a) introducing a pool of oligonucleotide probes to an obtained genomic sample to form target-probe complexes, wherein the pool of oligonucleotide probes comprise reference nucleic acid sequences capable of hybridizing to complementary nucleic acid sequences within the obtained genomic sample and wherein the oligonucleotide probes comprise a first member of a pair of specific binding entities; (b) flowing a solution including the formed target-probe complexes through a processing conduit of a microfluidic chip, wherein the processing conduit comprises a chamber including a plurality of beads, wherein the plurality of beads are functionalized with a second member of the pair of specific binding entities; (c) flowing at least one fluid through the processing conduit to remove off-target nucleic acids; and (d) flowing at least one reagent through the processing conduit to obtain the target nucleic acid sequences.

In some embodiments, the first moiety is biotin. In some embodiments, the second moiety is streptavidin.

In some embodiments, the obtained genomic sample is a sample derived from a mammalian subject, e.g. a human subject. In some embodiments, the obtained genomic sample is a blood sample or a blood plasma sample obtained from a mammalian subject, e.g. a human subject. In some embodiments, the obtained genomic sample is in the form of cell-free nucleic acids (e.g. having a size ranging from between about 180 bp to about 150 bp). In some embodiments, the obtained genomic sample in the form of cell-free nucleic acids comprises DNA and/or RNA.

In some embodiments, the method further comprises fragmenting the obtained genomic sample prior to the introduction of the pool of oligonucleotide probes.

In some embodiments, the flowing of the at least one fluid is sequentially repeated at least twice. In some embodiments, the flowing of the at least one fluid is sequentially repeated at least three times. In some embodiments, each sequentially flowed fluid is the same. In some embodiments, each sequentially flowed fluid is different. In some embodiments, the at least one fluid is a buffer.

In some embodiments, the at least one reagent is a Uracil-Specific Excision Reagent enzyme. In some embodiments, the at least one reagent is a buffer having a temperature ranging from between about 80° C. to about 105° C. In some embodiments, the at least one reagent is a buffer, and wherein the processing conduit is heated to a temperature ranging from between about 90° C. to about 100° C. In some embodiments, the reagent is an enzyme.

In some embodiments, the plurality of beads are non-magnetic beads. In some embodiments, the microfluidic chip comprises no mechanically moving parts. In some embodiments, the microfluidic chip is comprised of a non-magnetic material.

### BRIEF DESCRIPTION OF THE FIGURES

For a general understanding of the features of the disclosure, reference is made to the drawings. In the drawings, like reference numerals have been used throughout to identify identical elements.

FIG. 1A depicts a system including a microfluidic chip in communication with a fluidics module and a control system in accordance with one embodiment of the present disclosure.

FIG. 1B depicts a system including a microfluidic chip including a plurality of independently operable processing conduits in communication with a fluidics module and a control system in accordance with one embodiment of the present disclosure.

FIG. 1C depicts a system including a microfluidic chip including a processing conduit, wherein the processing conduit is in fluidic communication with a pump, a plurality of fluid and/or reagent reservoirs, a waste fluid/reagent vessel, a target collection vessel, and one or more conduits in accordance with one embodiment of the present disclosure.

FIG. 1D depicts a microfluidic chip having a plurality of independently operable processing conduits in a stacked configuration in accordance with one embodiment of the present disclosure.

FIG. 1E depicts a system including a microfluidic chip in fluidic communication with two pumps and a plurality of fluid and/or reagent reservoirs in accordance with one embodiment of the present disclosure.

FIG. 2A illustrates a top-down view of a processing conduit having a chamber fluidically coupled to a fluid inlet and a fluid outlet in accordance with one embodiment of the present disclosure.

FIG. 2B illustrates a top-down view of a processing conduit having a chamber fluidically coupled to a fluid inlet and a fluid outlet, wherein at least the chamber includes one or more posts in accordance with one embodiment of the present diclosure.

FIG. 2C illustrates a side cross-sectional view of the processing conduit of FIG. 2A.

FIG. 2D provides a side elevation view of a processing conduit having a chamber fluidically coupled to a fluid inlet and a fluid outlet in accordance with one embodiment of the present disclosure.

FIG. 2E provides an enlarged view of the chamber of the processing conduit of FIG. 2D.

FIG. 2F provides a side cross-sectional view of a chamber wall having an opening to permit the flow of fluid in accordance with one embodiment of the present disclosure.

FIG. 2G depicts a side cross-sectional view of a microfluidic chip having a chamber including a plurality of beads, where the beads have a diameter which is larger than one or more apertures within one or more walls of the chamber.

FIG. 2H depicts a cross-sectional view of a chamber of a processing conduit having one or more posts extending from one of a chamber ceiling and/or a chamber floor in accordance with one embodiment of the present disclosure.

FIG. 2I provides a cross-sectional view of a chamber of a processing conduit having one or more posts bridging a chamber ceiling and chamber floor in accordance with one embodiment of the present disclosure.

FIG. 3A illustrates a processing conduit having a chamber fluidically coupled to a fluid inlet and a fluid outlet in accordance with one embodiment of the present disclosure.

FIG. 3B illustrates a processing conduit having a chamber fluidically coupled to a fluid inlet and a fluid outlet, wherein at least the chamber includes one or more posts in accordance with one embodiment of the present disclosure.

FIG. 4A illustrates a processing conduit having a chamber fluidically coupled to a fluid inlet and a fluid outlet in accordance with one embodiment of the present disclosure.

FIG. 4B illustrates a processing conduit having a chamber fluidically coupled to a fluid inlet and a fluid outlet, wherein at least the chamber includes one or more posts in accordance with one embodiment of the present disclosure.

FIG. 4C illustrates a side cross-sectional view of the processing conduit of FIG. 4A.

FIG. 5A illustrates a processing conduit having two chambers fluidically coupled to one another through a transfer channel, wherein the two chambers are in fluidic communication with a fluid inlet and a fluid outlet in accordance with one embodiment of the present disclosure.

FIG. 5B illustrates a side cross-sectional view of the microfluidic chip of FIG. 5A.

FIG. 6A illustrates a processing conduit having two chambers fluidically coupled to one another through a serpentine transfer channel, wherein the two chambers are in fluidic communication with a fluid inlet and a fluid outlet in accordance with one embodiment of the present disclosure.

FIG. 6B illustrates a side cross-sectional view of the microfluidic chip of FIG. 6A.

FIG. 7A depicts a microfluidic chip including a plurality of independently operable processing conduits, wherein each processing conduit is in fluidic communication with a fluid inlet and a fluid outlet.

FIG. 7B depicts a microfluidic chip including a plurality of independently operable processing conduits, wherein each processing conduit is in fluidic communication with a fluid inlet and a fluid outlet, wherein at least the chambers of the processing conduits are illustrated as including one or more posts in accordance with one embodiment of the present disclosure.

FIG. 7C depicts a microfluidic chip including a plurality of independently operable processing conduits, wherein each processing conduit is in fluidic communication with a fluid inlet and a fluid outlet, wherein at least the chambers of the processing conduits are illustrated as including one or more posts in accordance with one embodiment of the present disclosure.

FIG. 7D depicts a microfluidic chip including a plurality of independently operable processing conduits, wherein each processing conduit includes two chambers and wherein the two chambers are fluidically coupled to one another through a serpentine transfer channel.

FIG. 8A sets forth a flowchart providing a method of purifying one or more types of molecule using the microfluidic devices of the present disclosure.

FIG. 8B sets forth a flowchart providing a method of purifying one or more types of molecule using the microfluidic devices of the present disclosure.

FIG. 9 sets forth a flowchart showing a method of enriching a solution with target molecules in accordance with one embodiment of the present disclosure.

FIG. 10 provides electropherograms of fluids collected after being flowed through a microfluidic device of the present disclosure.

FIG. 11A illustrates a method of bead capture and temperature-mediated release of target molecules hybridized to biotinylated oligonucleotides.

FIG. 11B illustrates the quantities of target recovered (as determined by quantitative polymerase chain reaction (qPCR)) as fluids are flowed through the microfluidic device (FT), after one or more wash solutions are flowed through the microfluidic device (W), and after eluent is collected (E).

FIG. 12A illustrates a method of bead capture and enzymatic release of target molecules hybridized to uracil-linked biotinylated oligonucleotides.

FIG. 12B illustrates the quantities of target recovered (as determined by qPCR) as fluids are flowed through the microfluidic device (FT), after one or more wash solutions are flowed through the microfluidic device (W), and after eluent is collected (E).

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins.

As used herein, the term “channel” refers to an enclosed passage within a microfluidic chip through which a fluid can flow. The channel can have one or more openings for introduction of a fluid. Each channel may include a coating, e.g. a hydrophilic or hydrophobic coating.

As used herein, the term “conjugate” refers to two or more molecules (and/or materials such as nanoparticles) that are covalently linked into a larger construct. In some embodiments, a conjugate includes one or more biomolecules (such as peptides, proteins, enzymes, sugars, polysaccharides, lipids, glycoproteins, and lipoproteins) covalently linked to one or more other molecules, such as one or more other biomolecules.

As used herein, the term “enrichment” refers to the process of increasing the relative abundance of a population of molecules, e.g. nucleic acid molecules, in a sample relative to the total amount of the molecules initially present in the sample before treatment. Thus, an enrichment step provides a percentage or fractional increase rather than directly increasing for example, the copy number of the nucleic acid sequences of interest as amplification methods, such as a polymerase chain reaction, would.

As used herein, the term “fluid” refers to any liquid or liquid composition, including water, solvents, buffers, solutions (e.g. polar solvents, non-polar solvents), and/or mixtures. The fluid may be aqueous or non-aqueous. Non-limiting examples of fluids include washing solutions, rinsing solutions, acidic solutions, alkaline solutions, transfer solutions, and hydrocarbons (e.g., alkanes, isoalkanes and aromatic compounds such as xylene).

In some embodiments, washing solutions include a surfactant to facilitate spreading of the washing liquids over the specimen-bearing surfaces of the slides. In some embodiments, acid solutions include deionized water, an acid (e.g., acetic acid), and a solvent. In some embodiments, alkaline solutions include deionized water, a base, and a solvent. In some embodiments, transfer solutions include one or more glycol ethers, such as one or more propylene-based glycol ethers (e.g., propylene glycol ethers, di(propylene glycol) ethers, and tri(propylene glycol) ethers, ethylene-based glycol ethers (e.g., ethylene glycol ethers, di(ethylene glycol) ethers, and tri(ethylene glycol) ethers), and functional analogs thereof.

Non-liming examples of buffers include citric acid, potassium dihydrogen phosphate, boric acid, diethyl barbituric acid, piperazine-N,N′-bis(2-ethanesulfonic acid), dimethylarsinic acid, 2-(N-morpholino)ethanesulfonic acid, tris(hydroxymethyl)methylamine (TRIS), 2-(N-morpholino)ethanesulfonic acid (TAPS), N,N-bis(2-hydroxyethyl)glycine(Bicine), N-tris(hydroxymethyl)methylglycine (Tricine), 4-2-hydroxymethyl-1-piperazineethanesulfonic acid (HEPES), 2-{[tris(hydroxymethyl)methyl]amino}ethanesulfonic acid (TES), and combinations thereof. In other embodiments, the buffer may be comprised of tris(hydroxymethyl)methylamine (TRIS), 2-(N-morpholino)ethanesulfonic acid (TAPS), N,N-bis(2-hydroxyethyl)glycine(Bicine), N-tris(hydroxymethyl)methylglycine (Tricine), 4-2-hydroxyethyl-1-piperazineethanesulfonic acid (HEPES), 2-{[tris(hydroxymethyl)methyl]amino}ethanesulfonic acid (TES), or a combination thereof. Additional wash solutions, transfer solutions, acid solutions, and alkaline solutions are described in United States Patent Application Publication No. 2016/0282374, the disclosure of which is hereby incorporated by reference herein in its entirety.

As used herein, “microfluidic” refers to a system or device having one or more fluidic channels, conduits, or chambers that are generally fabricated at the millimeter to nanometer scale. As such, a “microfluidic device,” as used herein, refers to any device that allows for the precise control and manipulation of fluids that are geometrically constrained to structures in which at least one dimension (width, length, height) may be less than 1 mm. In some embodiments, the microfluidic device includes a microfluidic chip including one or more channels and/or conduits.

As used herein, the phrase “next generation sequencing (NGS)” refers to sequencing technologies having high-throughput sequencing as compared to traditional Sanger- and capillary electrophoresis-based approaches, wherein the sequencing process is performed in parallel, for example producing thousands or millions of relatively small sequence reads at a time. Some examples of next generation sequencing techniques include, but are not limited to, sequencing by synthesis, sequencing by ligation, and sequencing by hybridization. These technologies produce shorter reads (anywhere from about 25 to about 500 bp) but many hundreds of thousands or millions of reads in a relatively short time. The term “next-generation sequencing” refers to the so-called parallelized sequencing-by-synthesis or sequencing-by-ligation platforms currently employed by Illumina, Life Technologies, and Helicos Biosciences. Next-generation sequencing methods may also include nanopore sequencing methods with electronic-detection (Oxford Nanopore and Roche Diagnostics).

As used herein, the term “nucleic acid” refers to a high-molecular-weight biochemical macromolecule composed of nucleotide chains that convey genetic information. The most common nucleic acids are deoxyribonucleic acid (DNA) and ribonucleic acid (RNA). The monomers from which nucleic acids are constructed are called nucleotides. Each nucleotide consists of three components: a nitrogenous heterocyclic base, either a purine or a pyrimidine (also known as a nucleobase); and a pentose sugar. Different nucleic acid types differ in the structure of the sugar in their nucleotides; DNA contains 2-deoxyribose while RNA contains ribose.

As used herein, the term “plurality” refers to two or more, for example, 3 or more, 4 or more, 5 or more, etc.

As used herein, a “reaction” between any two different reactive groups (such as any two reactive groups of a reagent and a particle) may mean that a covalent linkage is formed between the two reactive groups (or two reactive functional groups); or may mean that the two reactive groups (or two reactive functional groups) associate with each other, interact with each other, hybridize to each other, hydrogen bond with each other, etc. In some embodiments, the “reaction” includes binding events, e.g. binding events between reactive function groups or binding events between first and second members of a pair of specific binding entities.

As used herein, the term “reagent” refers to solutions or suspensions including one or more agents capable of covalently or non-covalently reacting with, coupling with, interacting with, or hybridizing to another entity. Non-limiting examples of such agents include specific-binding entities, antibodies (primary antibodies, secondary antibodies, or antibody conjugates), nucleic acid probes, oligonucleotide sequences, detection probes, chemical moieties bearing a reactive functional group or a protected functional group, enzymes, solutions or suspensions of dye or stain molecules.

As used herein, the term “sequencing” refers to biochemical methods for determining the order of the nucleotide bases, adenine, guanine, cytosine, and thymine, in a DNA oligonucleotide. Sequencing, as the term is used herein, can include without limitation parallel sequencing or any other sequencing method known of those skilled in the art, for example, chain-termination methods, rapid DNA sequencing methods, wandering-spot analysis, Maxam-Gilbert sequencing, dye-terminator sequencing, or using any other modern automated DNA sequencing instruments.

As used herein, the term “specific binding entity” refers to a member of a specific-binding pair. Specific binding pairs are pairs of molecules that are characterized in that they bind each other to the substantial exclusion of binding to other molecules (for example, specific binding pairs can have a binding constant that is at least 10³ M⁻¹ greater, 10⁴ M⁻¹ greater or 10⁵ M⁻¹ greater than a binding constant for either of the two members of the binding pair with other molecules in a biological sample). Particular examples of specific binding moieties include specific binding proteins (for example, antibodies, lectins, avidins such as streptavidins, and protein A). Specific binding moieties can also include the molecules (or portions thereof) that are specifically bound by such specific binding proteins.

As used herein, the term “substantially” means the qualitative condition of exhibiting total or near-total extent or degree of a characteristic or property of interest. In some embodiments, “substantially” means within about 5%. In some embodiments, “substantially” means within about 10%. In some embodiments, “substantially” means within about 15%. In some embodiments, “substantially”means within about 20%.

As used herein, the terms “target” or “target sequence” refer to nucleic acid sequences of interest, e.g. those which hybridize to oligonucleotide probes.

### Overview

The present disclosure is directed to automated systems including a microfluidic chip having one or more independently operable processing conduits. In some embodiments, the automated systems are suitable for use in sample cleanup and/or target enrichment processes, such as sample cleanup and/or target enrichment processes conducted prior to sequencing a sample using next-generation sequencing. In some embodiments, the automated systems are also suitable for purifying solutions and/or performing solid-phase synthesis. As noted herein, in some embodiments, the microfluidic devices do not rely on magnetic separation processes, utilize magnetic beads, and/or include magnetic components.

### Microfluidic Device

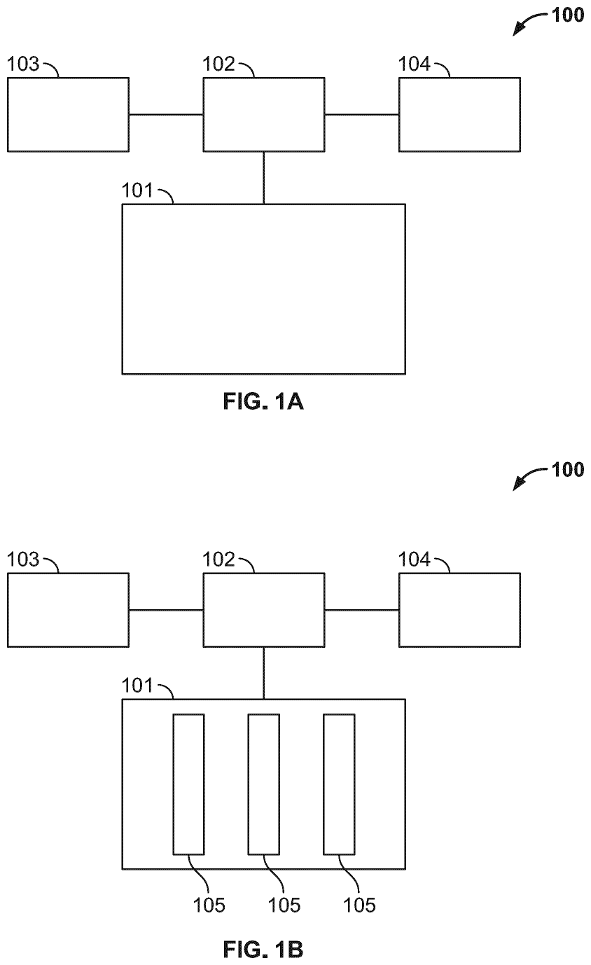

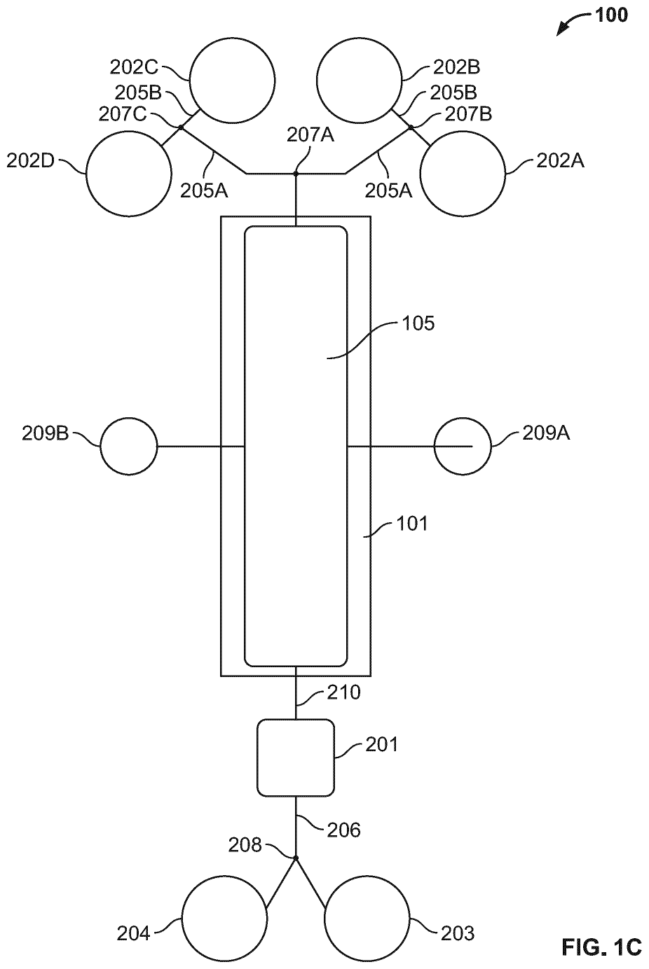

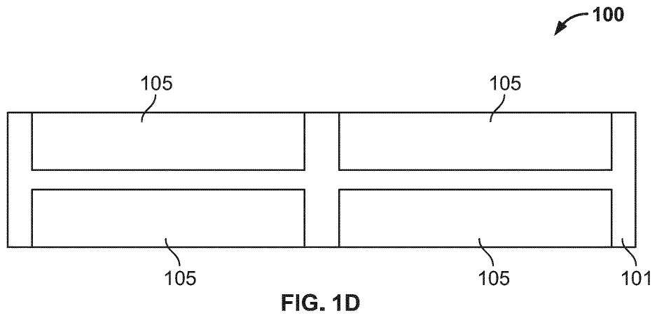

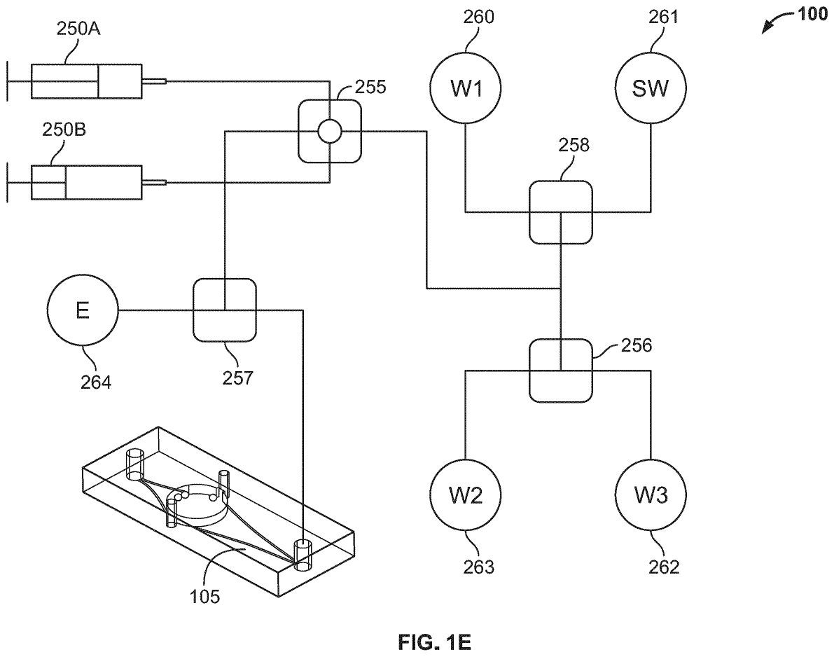

In one aspect of the present disclosure are microfluidic devices including a microfluidic chip having one or more independently operable processing conduits. With reference to FIGS. 1A-E, in one aspect of the present disclosure is a microfluidic device 100 including a fluidics module 102, a control system 104, and a microfluidic chip 101.

In some embodiments, the microfluidic devices 100 are fluidically coupled to one or more fluid and/or reagent reservoirs. In some embodiments, the microfluidic devices 100 are in further communication with one or more sensors, heating and/or cooling modules, and/or upstream and/or downstream processing systems, e.g. sequencing devices, instruments for conducting polymerase chain reactions, chemical analyzers, detectors, etc. Microfluidic devices and the components constituting any microfluidic device (e.g. control systems, pumps, valves, etc.) are described in further detail herein.

### Microfluidic Chip

The microfluidic devices 100 of the present disclosure include a microfluidic chip 101 having one or more independently operable processing conduits 105. In some embodiments, the microfluidic chip 101 includes between 1 and 600 independently operable processing conduits 105. In other embodiments, the microfluidic chip 101 includes between 1 and 500 independently operable processing conduits 105. In yet other embodiments, the microfluidic chip 101 includes between 1 and 400 independently operable processing conduits 105. In further embodiments, the microfluidic chip 101 includes between 1 and 300 independently operable processing conduits 105. In some embodiments, the microfluidic chip 101 includes one independently operable processing conduit 105. In other embodiments, the microfluidic chip 101 includes two or more independently operable processing conduits 105. In yet other embodiments, the microfluidic chip 101 includes three or more independently operable processing conduits 105. In further embodiments, the microfluidic chip 101 includes five or more independently operable processing conduits 105. In yet further embodiments, the microfluidic chip 101 includes ten or more independently operable processing conduits 105.

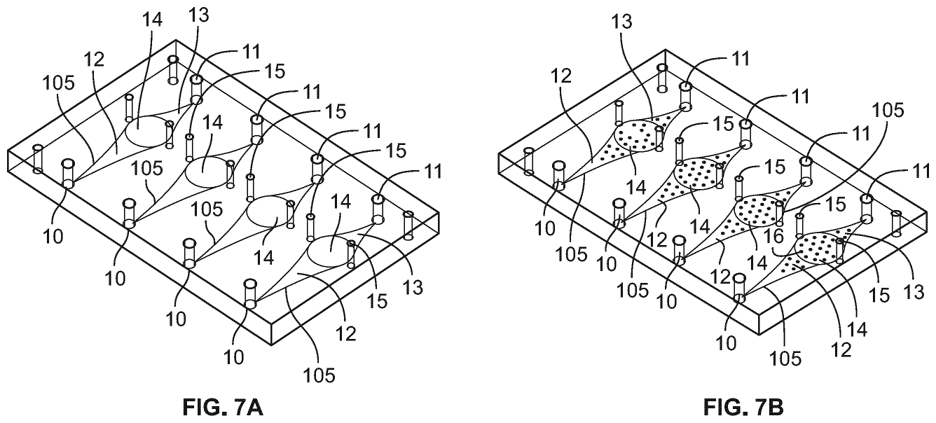

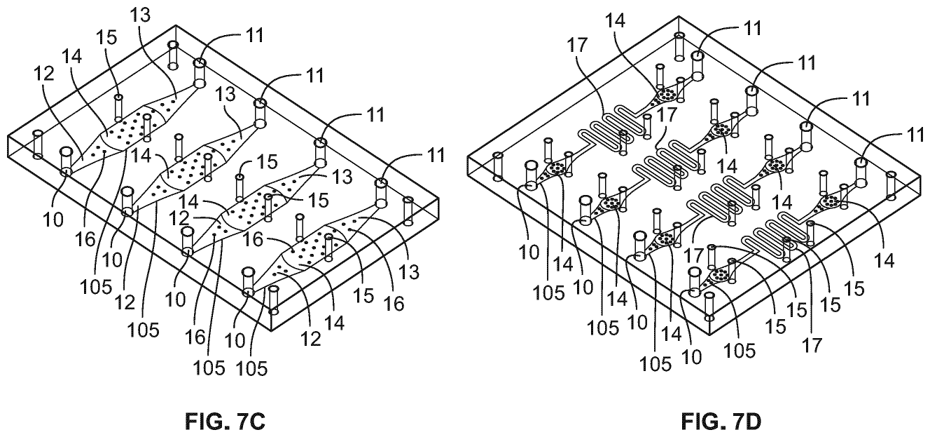

In those embodiments where two or more independently operable processing conduits 105 are included within any microfluidic chip, the processing units may be arranged within the same plane. For example, FIG. 1B illustrates three independently operable processing conduits 105 arranged parallel to one another and within the same plane. Likewise, FIGS. 7A-7D illustrate a plurality of independently operable processing conduits 105 arranged parallel to one another and in the same plane. In other embodiments, two or more processing conduits 105 may be arranged in different planes. For example, in some embodiments, two or more independently operable processing conduits 105 may be at least partially stacked over each other within any microfluidic chip 101 (see, for example, FIG. 1D).

The skilled artisan will appreciate that, in some embodiments, each independently operable processing conduit may be coupled its own set of reservoirs, conduits, pumps, etc. In other embodiments, two or more of the independently operable processing conduits may be coupled to shared fluid reservoirs and/or shared reagent reservoirs. As described herein, fluid from shared reservoirs may be supplied to each independently operable processing conduit by controlling one or more valves disposed within the reservoirs themselves or within channels or conduits coupling the reservoirs to the independently operable processing conduits. Likewise, fluid from shared reservoirs may be supplied to the independently operable processing conduits through the action of one or more pumps in fluidic communication with each independently operable processing conduit.

### Processing Conduit

Examples of independently operable processing conduits having different configurations are described herein. While different configurations are described, each processing conduit serves the purpose of allowing fluids and/or reagents introduced at an inlet to be passed through an inlet channel and into one or more chambers in fluidic communication with the inlet channel. The introduced fluids and/or reagents are then permitted to flow out of the one or more chambers and through an outlet channel in communication therewith. Ultimately, the fluid and/or reagents flowed through the processing conduit are collected and/or analyzed after passing through an outlet fluidically coupled to the outlet channel.

As described herein, the flow of fluid through the processing conduit may be controlled using one or more valves and/or one or more pumps in fluidic communication with the processing conduit (valves and pumps are described further herein). In some embodiments, the processing conduits do not include any moving parts. Nor are the processing conduits coupled to magnets, magnetic strips, and/or magnetic components. As such, any non-magnetic beads provided within the one or more chambers of the processing conduits are moved (e.g. flowed) through the processing conduit solely through the movement (e.g. flow) of fluids and/or reagents (e.g. via the action of one or more pumps communicatively coupled thereto). In some embodiments, the processing conduits do not rely on magnetic separation.

Each of the independently operable processing conduits include one or more chambers. In some embodiments, the independently operable processing conduits include between 1 and about 100 chambers. In some embodiments, the independently operable processing conduits include between 1 and about 50 chambers. In some embodiments, the independently operable processing conduits include between 1 and about 20 chambers. In some embodiments, the independently operable processing conduits include between 1 and about 10 chambers. In other embodiments, the independently operable processing conduits include between 1 and 5 chambers. In yet other embodiments, the independently operable processing conduits include between 1 and 3 chambers.

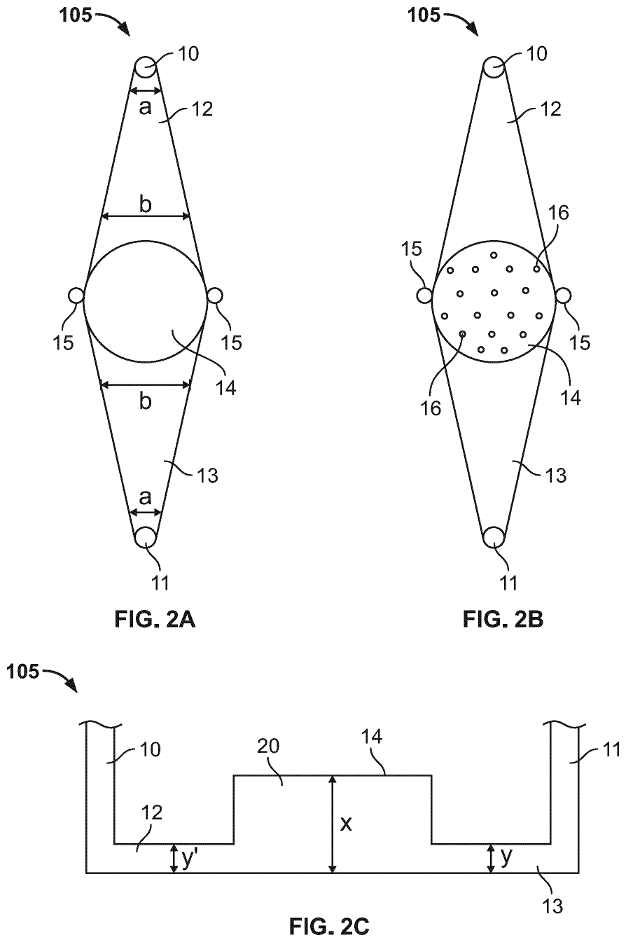

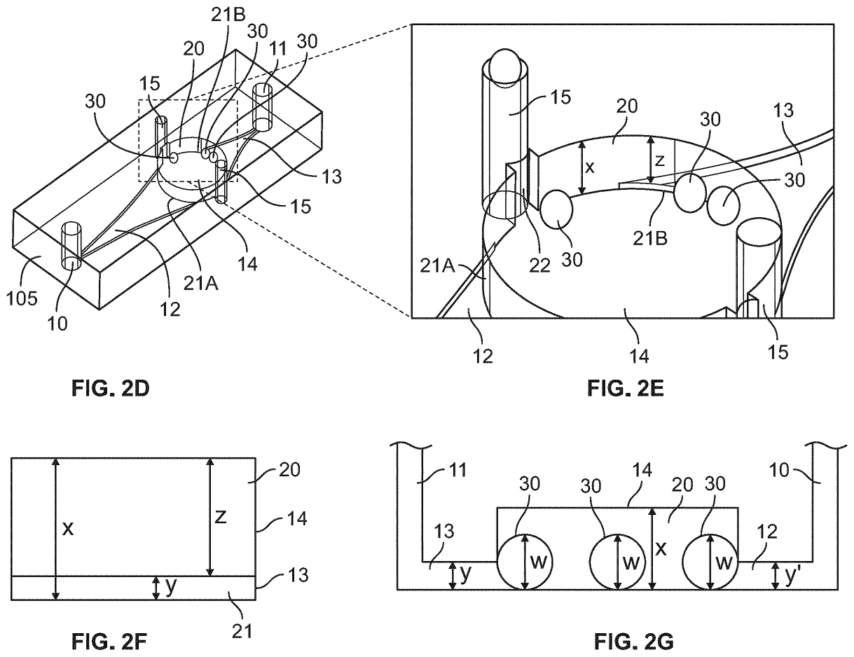

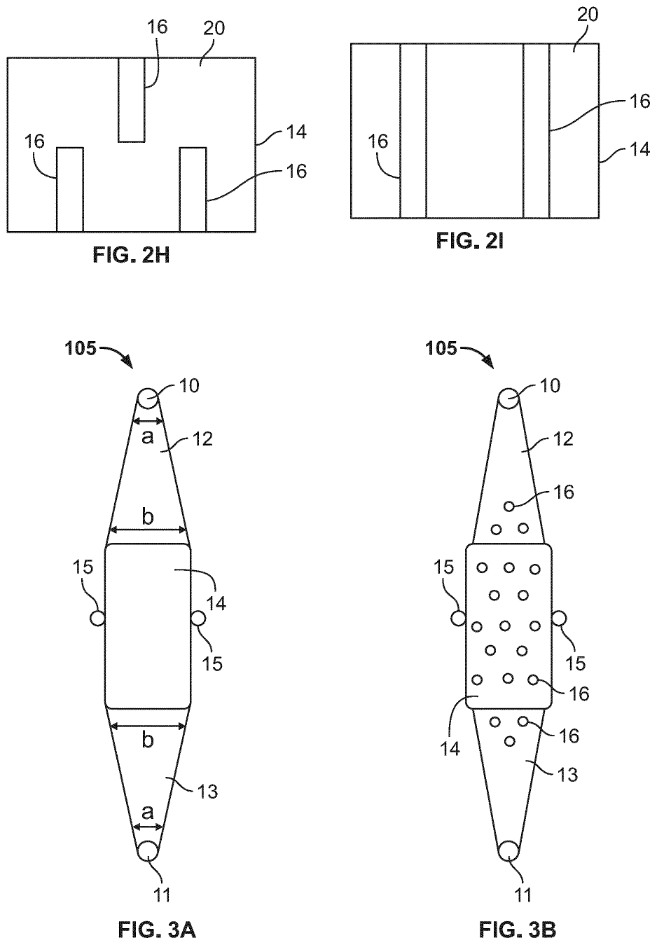

In some embodiments, the processing conduits 105 include a single chamber. By way of illustration, FIG. 2A depicts a processing conduit 105 having a chamber 14 fluidically coupled to an inlet channel 12 and an outlet channel 13. In some embodiments, the inlet channel 12 is in fluidic communication with an inlet 10; and the outlet channel 13 is in communication with an outlet 11.

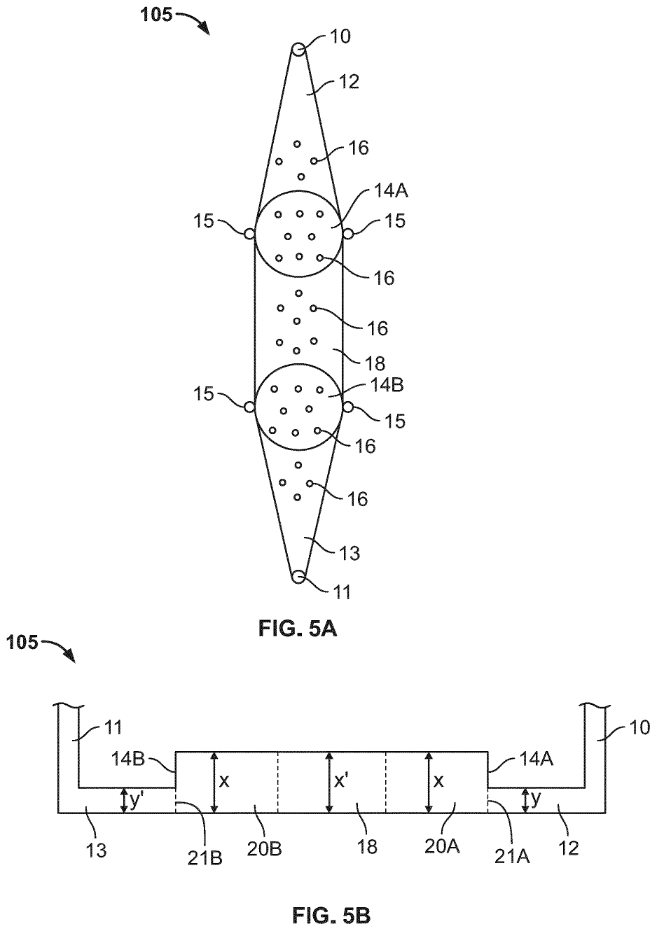

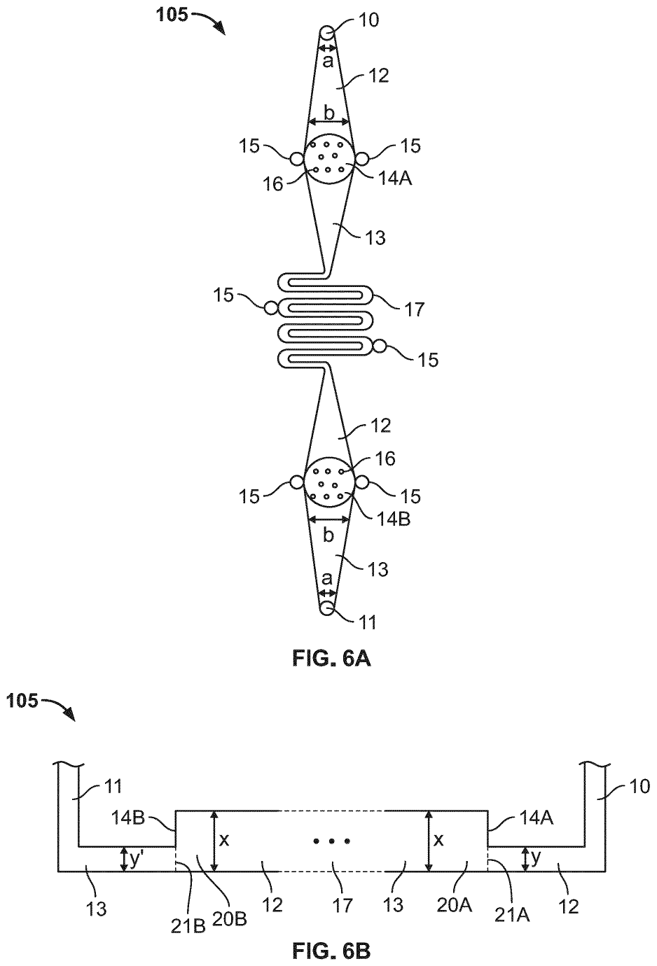

In other embodiments, the processing conduits 105 include two or more chambers. By way of illustration, FIGS. 5A and 6A depict processing conduits 105 having channels 14A and 14B, whereby the channels 14A and 14B are coupled to one another through a transfer channel. The transfer channel may have any size or shape. For example, and as depicted in FIG. 5A, the transfer channel 18 may be linear. By way of another example, and as depicted in FIG. 6A, the transfer channel 17 may have a serpentine shape which, it is believed, allows for an increased surface area as compared with a linear transfer channel. In some embodiments, the first chamber 14A is in fluidic communication with an inlet channel 12; while the second chamber 14B is in fluidic communication with an outlet channel 13. In some embodiments, the inlet channel 12 is in fluidic communication with an inlet 10; and the outlet channel 13 is in communication with an outlet 11.

The chambers 14 of the processing conduits 105 may have any size or shape. In some embodiments, the chambers are circular (see, e.g., FIG. 2A). In other embodiments, the chambers are ovoid or substantially ovoid (see, e.g., FIG. 7C). In yet other embodiments, the chambers are rectangular or substantially rectangular (see, e.g., FIG. 3A).

In some embodiments, a volume of a chamber ranges from between about 0.1 μL to about 10 mL. In other embodiments, a volume of a chamber ranges from between about 0.1 μL to about 7.5 mL. In other embodiments, a volume of a chamber ranges from between about 0.1 μL to about 5 mL. In yet other embodiments, a volume of a chamber ranges from between about 0.1 μL to about 2.5 mL. In some embodiments, an area of a bottom surface of the chamber ranges from between about 1 mm² to about 100 cm². In some embodiments, an area of a bottom surface of the chamber ranges from between about 1 cm² to about 100 cm². In some embodiments, an area of a bottom surface of the chamber ranges from between about 1 cm² to about 50 cm². In other embodiments, an area of a bottom surface of the chamber ranges from between about 1 mm² to about 500 mm². In other embodiments, an area of a bottom surface of the chamber ranges from between about 1 mm² to about 100 mm².

In some embodiments, the chamber is configured to permit the introduction and/or removal of a plurality of beads. In some embodiments, the beads are non-magnetic beads. Examples of suitable non-magnetic beads include silica beads, alginate hydrogel beads, agarose hydrogel beads, poly(N-isopropylacrylamide) (NIPAM) gel beads, cellulose beads, polyethylene (PE) beads, polypropylene (PP) beads, polymethyl methacrylate (PMMA) beads, nylon (PA) beads, polyurethane beads, acrylates copolymer beads, polyquaterniums beads, polysorbate beads, and polyethylene glycol (PEG) beads (any of which may be functionalists or further derivatized before or after introduction to a chamber). In some embodiments, the beads have an average diameter ranging from between about 0.1 μm to about 5 mm. In some embodiments, the beads have an average diameter ranging from between about 0.1 mm to about 1 mm. In yet other embodiments, the beads have an average diameter ranging from between about 0.1 mm to about 1 mm.

In some embodiments, the chamber may be in fluidic communication with one or more ducts which facilitate the introduction and/or removal of the plurality of beads from the chamber. In some embodiments, the chamber is in fluidic communication with two ducts. In other embodiments, the chamber is in fluidic communication with three or more ducts. In yet other embodiments, the chamber is in fluidic communication with four or more ducts.

FIGS. 2A, 3A, and 4A depict a chamber 14 in fluidic communication with two ducts 15, where the two ducts 15 are arranged about 180-degrees from one another. In some embodiments, one of the two ducts 15 is configured to allow for the introduction of beads while the other of the two ducts 15 is configured to allow for the removal of beads. In some embodiments, each of the ducts 15 may be in fluidic communication with a bead transfer conduit, a bead source (such as a bead storage vessel or a bead collection vessel), one or more valves, and/or one or more pumps.

In those embodiments that include two or more chambers, each one of the two or more chambers may be in fluidic communication with one or more ducts (see, for example, FIGS. 5A and 6A). In some embodiments, the ducts include one or more doors or valves which permit the ducts to be closed. In some embodiments, once the chamber is loaded with a pre-loaded with a predetermined number of beads, the doors or valves within the ducts may be closed such that the beads are sealed within the chamber. The doors or valves may later be opened to recover the beads.

With reference to FIG. 2E, the ducts 15 may be external to the chamber 14. In some embodiments, a wall 20 of chamber 14 may include a ductal opening 22 which permits passage of the beads from the one or more ducts 15 into the chamber 14. In some embodiments, the ductal opening 22 may have any size and/or shape provided that it allows at least one bead to pass into or out of the chamber.

In some embodiments, the chamber is adapted such that any introduced beads may move within the chamber but not leave the chamber. With reference to FIGS. 2D and 2E, in some embodiments, a wall 20 of the chamber 15 includes first and second apertures 21A and 21B through which fluids and/or reagents may flow, but not any of the beads introduced into the chamber 14. FIG. 2E illustrates a cross-sectional view of a chamber 14 showing a wall 20 and an aperture 21 within the wall. FIGS. 2F and 2G illustrates a cross-sectional view of a processing conduit 105 and depicts a plurality of beads 30 each having an average diameter “w” which is less than a height “x” of wall 20, but greater than a height “y” of aperture 21. In this manner, fluids and/or reagents (and any target molecules and/or particles) may flow through the processing conduit 105 from the inlet channel 12, into the chamber 14, and out of the outlet channel 13, but where the plurality of beads 30 are retained within chamber 14 during the flow of the fluids and/or reagents.

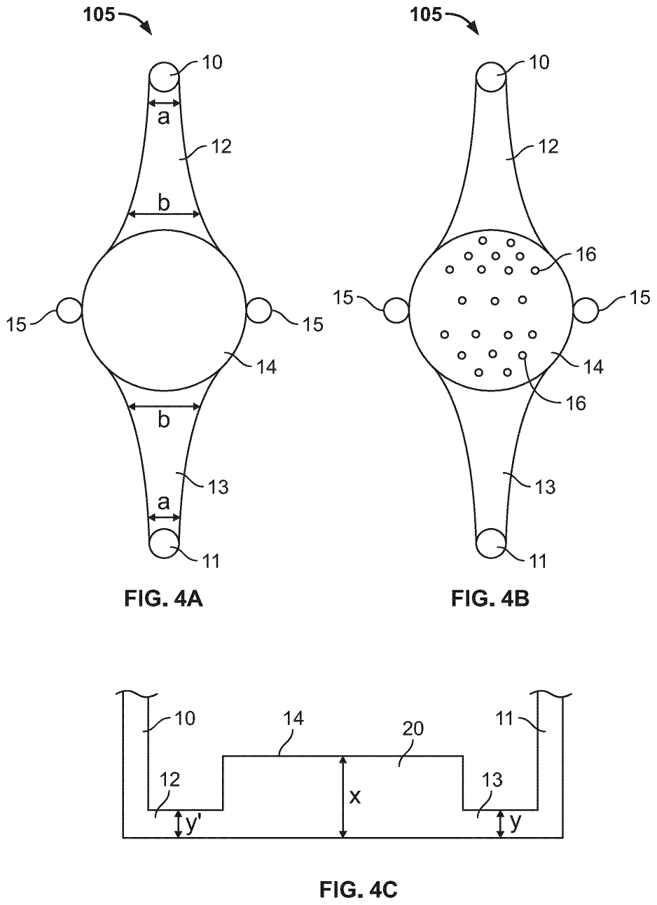

With reference to FIG. 2C, in some embodiments, a height of a chamber is greater than a height of at least one of an inlet channel or an outlet channel. For example, and as illustrated in FIGS. 2C and 4C, a height “x” of the chamber 14 may be greater than a height “y” of either the inlet channel 12 or a height “y” of the outlet channel 13. In some embodiments, a height “x” of the chamber 14 ranges from between 0.1 μm to about 10 cm. In other embodiments, a height “x” of the chamber 14 ranges from between 0.1 mm to about 1 cm. In yet other embodiments, a height “x” of the chamber 14 ranges from between 0.1 mm to about 1 mm. In some embodiments, a height “x” of the outlet channel 13 and/or inlet channel 12 ranges from between 0.1 μm to about 10 cm. In other embodiments, a height “x” of the outlet channel 13 and/or inlet channel 12 ranges from between 0.1 mm to about 1 cm. In yet other embodiments, a height “x” of the outlet channel 13 and/or inlet channel 12 ranges from between 0.1 mm to about 1 mm.

In those embodiments where two or more channels are fluidically coupled to one another via transfer channel, any beads introduced into a first chamber may be permitted to flow from a first chamber to one or more additional chambers. In addition, the beads, once flowed from the first chamber to another of the one or more additional chambers, may be allowed to flow back into the first chamber. In these embodiments, a chamber wall in communication with the transfer channel includes an aperture which permits the transfer of the beads from the chamber to the transfer channel. Alternatively, a chamber may not include any wall in an area where the chamber joins the transfer channel, which again permits the transfer of the beads from the chamber to the transfer channel.

For example, and with reference to FIG. 5A, a processing conduit 105 may include (i) a first chamber 14A having a first wall 20A including a first aperture 21A, where the first wall 20A is in communication with the inlet channel 12; (ii) a second chamber 14B having a second wall 20B including a second aperture 21B in communication with the outlet channel 13; and wherein the chambers 14A and 14B do not include walls or wall portions in those regions of the chamber which are in communication with the transfer channel 18.

In some embodiments, the inlet and outlet channels 12 and 13, respectively, may have any size and/or shape. In some embodiments, the inlet channel 12 includes linear side walls, such as depicted in FIGS. 2A, 2B, 3A, and 3B. In other embodiments, the inlet and outlet channels 12 and 13, respectively, include curvilinear or arcuate side walls, such as depicted in FIGS. 2D, 4A, and 4C.

In some embodiments, the inlet and outlet channels 12 and 13, respectively, taper from a first width where they join the chamber to a second width where they join the inlet and outlet 10 and 11, respectively. In some embodiments, the inlet channel 12 tapers from a first width where it joins the chamber 14 to a second width where it joins the inlet 10, where the first width is greater than the second width. For example, FIGS. 2A, 3A, and 4A each depict an inlet channel 12 which increasingly tapers in width from the inlet 10 to the chamber 14.

With reference to FIGS. 2A and 3A, in some embodiments, a width “a” of the inlet channel 12 ranges from between 0.1 μm to about 10 cm. In other embodiments, a width “a” of the inlet channel 12 ranges from between 1 mm to about 10 cm. In other embodiments, a width “a” of the inlet channel 12 ranges from between 1 mm to about 1 cm. In other embodiments, a width “a” of the inlet channel 12 ranges from between 1 mm to about 5 mm. In some embodiments, a width “b” of the inlet channel 12 ranges from between 0.1 μm to about 10 cm. In other embodiments, a width “b” of the inlet channel 12 ranges from between 1 mm to about 10 cm. In other embodiments, a width “b” of the inlet channel 12 ranges from between 1 mm to about 1 cm. In other embodiments, a width “b” of the inlet channel 12 ranges from between 1 mm to about 5 mm. It is believed that tapered channels substantially mitigate the trapping of air and/or assist in stabilizing and/or developing a fluid profile.

In some embodiments, the outlet channel 13 tapers from a first width where it joins the chamber 14 to a second width where it joins the outlet 11. For example, FIGS. 2A, 3A, and 4A each depict an outlet channel 13 which decreasingly tapers in width from the chamber 14 to the outlet 11. In some embodiments, a width “a” of the outlet channel 13 ranges from between 0.1 μm to about 10 cm. In other embodiments, a width “a” of the outlet channel 13 ranges from between 1 mm to about 10 cm. In some embodiments, a width “a” of the outlet channel 13 ranges from between 1 mm to about 50 mm. In other embodiments, a width “a” of the outlet channel 13 ranges from between 1 mm to about 10 mm. In some embodiments, a width “b” of the outlet channel 13 ranges from between 0.1 um to about 10 cm. In other embodiments, a width “b” of the outlet channel 13 ranges from between 1 mm to about 10 cm. In some embodiments, a width “b” of the outlet channel 13 ranges from between 1 mm to about 50 mm. In other embodiments, a width “b” of the outlet channel 13 ranges from between 1 mm to about 10 mm.

In some embodiments, at least one of the one or more chambers, inlet channels, and/or outlet channels of a processing conduit includes one or more posts. It is believed the inclusion of the one or more posts introduce turbulence into the flow of the fluids and/or reagents passing through the chambers, inlet channels, and outlet channels, thereby facilitating mixing between the introduced fluids, reagents, and/or beads. It is believed that such a chaotic microenvironment enhances the mixing rate by introducing advective molecular transport and exchange between two or more different subjects.

In some embodiments, the number of posts within the chamber ranges from between 1 to about 1,000,000. In other embodiments, the number of posts within the chamber ranges from between 1 to about 1,0,000. In some embodiments, the number of posts within the chamber ranges from between 1 to about 1,000. In other embodiments, the number of posts within the chamber ranges from between 1 to about 100. In some embodiments, the number of posts within the inlet channel and/or the outlet channel ranges from between 1 to about 500. In other embodiments, the number of posts within the inlet channel and/or the outlet channel ranges from between 1 to about 250. In other embodiments, the number of posts within the inlet channel and/or the outlet channel ranges from between 1 to about 100.

For example, an as illustrated in FIGS. 2B and 4B, the chamber 14 includes a plurality of posts 16. By way of another example, and as depicted in FIG. 3B, the chamber 14, the inlet channel 12, and the outlet channel 13, each include a plurality of posts 16. By way of yet another example, FIG. 5A depicts a processing conduit 105 including first and second chambers 14A and 14B fluidically coupled to one another through a transfer channel 18, where the first chamber 14A, the second chamber 14B, and the transfer channel 18 each include a plurality of posts.

In some embodiments, the one or more posts extend from the floor or the ceiling of the chamber, inlet channel, and/or outlet channel (see, for example, FIG. 2H). In other embodiments, the one or more posts extend from the floor to the ceiling of the chamber, inlet channel, and/or outlet channel (see, for example, FIG. 2I). In some embodiments, the posts may have any size and shape, such as cylindrical or polygonal. In some embodiments, the posts may have any size and/or shape. For example, the posts may be cylindrical or rectangular. In some embodiments, the posts have a diameter ranging from between about 0.1 μm to about 1 mm. In some embodiments, the posts have a diameter ranging from between about 0.5 μm to about 1 mm.

### Reservoirs and Vessels

The microfluidic device 100 may be fluidically coupled to any number of reagent reservoirs, bead storage vessels, bead collection vessels, fluid reservoirs, waste collection reservoirs, etc. In some embodiments, the microfluidic device includes a separate fluid and/or reagent reservoir for each different fluid and/or reagent for introduction into the processing conduit 105. As noted herein, the reservoirs may be shared among two or more independently operable processing conduits.

In some embodiments, each of the reservoirs may be fluidically coupled to the microfluidic device 100 via a conduit as described herein. For example, FIG. 1C illustrates four fluid and/or reagent reservoirs 202A-202D fluidically coupled to an inlet of the processing conduit 105. The skilled artisan will appreciate that one of the four fluid and/or reagent reservoirs 202A-202D depicted in FIG. 1C may be a sample reservoir where a sample to be purified or enriched is stored prior to its introduction into the processing conduit 105. In some embodiments, the volume of a fluid and/or reagent reservoir ranges from between about 10 μL to about 10 mL. In some embodiments, the volume of a fluid and/or reagent reservoir ranges from between about 1 mL to about 5 mL.

In some embodiments, the microfluidic device 100 may be fluidically coupled to one or more bead storage 209A or collection vessels 209B. In some embodiments, beads may be introduced to one or more chambers of a processing conduit through a conduit coupling a bead storage vessel to a first duct of the processing conduit. Likewise, in some embodiments, beads may be transferred from one or more chambers of a processing conduit through a conduit coupling a bead collection vessel to a second duct of the processing conduit. In some embodiments, the volume of a bead storage or collection vessel ranges from between about 0.1 μL to about 5 mL. In other embodiments, the volume of bead storage or collection vessel ranges from between about 0.1 mL to about 1 mL. In some embodiments, beads may be introduced into the microfluidic device through a bead packing inlet connected to a bead storage vessel using a pipette or syringe. Alternatively, if there is only one aperture in the wall of the chamber existing on the outlet side, beads can be introduced through the fluid inlet. In this particular embodiment, this would allow for the elimination of one or more ducts.

### Fluidics Module

The microfluidic devices 100 of the present disclosure also include a fluidics module comprising one or more conduits, one or more pumps, one or more valves, etc.

### Conduits

The microfluidic device 100 may include any number of conduits to facilitate the transfer of fluids, reagents, and/or beads to the inlet 10, outlet 12, and/or the ducts 15 of the processing conduit 105 of the microfluidic device 100. In some embodiments, each fluid and/or reagent for introduction to the processing conduit 105 may be stored in a separate fluid reservoir and/or reagent reservoir and wherein each fluid reservoir and/or reagent reservoir is independently coupled to a fluid transfer conduit or a reagent transfer conduit in fluidic communication with the inlet 10 of the processing conduit 105. In this manner, a reagent from a single reagent reservoir may be transferred via a reagent transfer conduit to the processing conduit 105. Likewise, a fluid from a single fluid reservoir may be transferred via a fluid transfer conduit to the processing conduit 105. In some embodiments, each of the fluid and/or reagent reservoirs and/or the fluid and/or reagent transfer conduits may include a valve, e.g. a 2-way valve, such that fluids and/or reagents may be flowed into the processing conduit 105, as described herein.

In some embodiments, and with reference to FIG. 1C, an inlet of a processing conduit 105 may be fluidically coupled to a branched conduit 205A, where each branch of the branched conduit 205A is fluidically coupled to a fluid transfer conduit 205B. In some embodiments, each fluid transfer conduit 205B is coupled to a fluid and/or reagent reservoir 202. In FIG. 1C, the processing conduit is illustrated as being in fluidic communication with four fluid and/or reagent reservoirs 202A-202D. As the skilled artisan will appreciate, each of the four fluid and/or reagent reservoirs 202A-202D of FIG. 1C may include a different fluid and/or a different reagent.

In some embodiments, the microfluidic device 100 may include one or more pumping conduits 210, where such pumping conduits serve to fluidically couple one or more pumps to processing conduit 205. In some embodiments, the microfluidic device 100 may include a waste transfer conduit 206 in fluidic communication with (i) the outlet of the processing conduit 105, (ii) and a waste fluid/reagent vessel 203 and/or a sample collection vessel 204.

### Valves

The microfluidic device 100 of the present disclosure may include one or more valves and/or microvalves. In some embodiments, the valves may be disposed within any conduit of the microfluidic device 100, with any portion of a conduit of the microfluidic device 100, or at a junction of any two conduits of the microfluidic device 100. In some embodiments, each of the valves of the microfluidic device 100 includes one or more ports, e.g. 1-port, 2-ports, or 3-ports.

Any type of valve may be utilized provided that the valve allows the flow of fluid, reagents, and/or beads throughout the microfluidic device 100 to be regulated, e.g. starting/stopping fluid flow, controlling the quantities of fluid flow, etc. In some embodiments, the valves are controlled based on signals from a control system 104, e.g. the control system 104 may command a valve to actuate to a first position, to a second position, or a third position such that fluid, reagent, and/or bead transfer may be regulated. Non-limiting examples of suitable microfluidic valves are described in U.S. Pat. No. 10,197,188; in U.S. Patent Publication Nos. 2008/0236668 and 2006/0180779; and in PCT Publication No. WO/2018/104516, the disclosures of which are hereby incorporated by reference herein in their entireties.

In some embodiments, and again with reference to FIG. 1C, one or more valves 207A, 207B, and 207C may be disposed in the branched conduit 205A and/or in the fluid transfer conduit 205B such that the flow of fluids and/or reagents from the reservoirs may be independently controlled. Alternatively, in other embodiments, each of the fluid and/or reagent reservoirs include a valve such that the flow of fluids and/or reagents from the reservoirs may be independently controlled. In some embodiments, one or more valves may be disposed within the waste transfer conduit 206.

### Pumps

In some embodiments, the microfluidic device 100 is in fluidic communication with one or more pumps. In some embodiments, the microfluidic device is in fluidic communication with two pumps. In other embodiments, the microfluidic device is in fluidic communication with three pumps. In yet other embodiments, the microfluidic device is in fluidic communication with four or more pumps.

In some embodiments, the one or more pumps facilitate the movement of fluid, reagents, and/or beads within the chambers, channels, and/or conduits of the microfluidic device. Any pump may be utilized within the microfluidic device of the present disclosure provided that the pump selected allows for control of the volume loaded into or discharged from the microfluidic device. In some embodiments, the one or more pumps are pressure pumps. In other embodiments, the one or more pumps are piezo-electric pumps. In some embodiments, the one or more pumps are peristaltic pumps. In some embodiments, the one or more pumps are syringe pumps. In some embodiments, the one or more pumps are volumetric pumps.

In some embodiments, the one or more pumps of the present disclosure have a volume ranging from between about 1 mL to about 100 mL. In other embodiments, the one or more pumps of the present disclosure have a volume ranging from between about 10 mL to about 100 mL. In some embodiments, the one or more pumps of the present disclosure may deliver a flow rate of between about 1 μL/minute to about 1000 mL/minute. In other embodiments, the one or more pumps of the present disclosure may deliver a flow rate of between about 10 μL/minute to about 500 mL/minute. In yet other embodiments, the one or more pumps of the present disclosure may deliver a flow rate of between about 10 μL/minute to about 100 mL/minute.

In some embodiments, each of the one or more pumps of the microfluidic device 100 are provided for a single purpose, e.g. infusing fluids, withdrawing fluids, transferring beads into and/or out of the one or more chambers. In other embodiments, any single pump may be used for multiple purposes. For example, one pump may facilitate both infusion and withdrawal of fluid.

In some embodiments, the microfluidic device is in communication with one or more of a “fluid injection pump” or “fluid withdrawal pump.” A “fluid infusion pump,” as used herein, refers to any device through which a fluid and/or reagent may be introduced into a microfluidic device, including into any of the chambers, channels, or conduits of the microfluidic devices of the present disclosure. As such, a fluid infusion pump can be used to deliver any fluid and/or reagent to any chamber, channel, and/or conduit; and/or any beads included within the fluid may be moved from one chamber of the microfluidic device 100 to another (such as through a transfer channel) through the actions of the fluid injection pump.

A “fluid withdrawal pump,” as used herein, refers to any device through which fluid may be removed from a microfluidic device, including from any of the chambers, channels, or conduits of the microfluidic devices of the present disclosure, or from any one or more of fluid reservoirs and/or reagent reservoirs in fluidic communication therewith. As such, a fluid withdrawal pump can be used to remove any fluid or reagent from any chamber, channel, conduit and/or reservoir; and any beads included within the fluid may be moved from one chamber of the microfluidic device 100 to another (such as through a transfer channel) through the actions of the fluid withdrawal pump.

In some embodiments, the one or more pumps are micropumps. In some embodiments, the micropumps are mechanical pumps (e.g. diaphragm micropumps and peristaltic micropumps). In some embodiments, the micropumps are non-mechanical pumps (e.g. valveless micropumps, capillary pumps, and chemically powered pumps). Devices are known for through pumping of small fluid quantities. For example, U.S. Pat. Nos. 5,094,594, 5,730,187 and 6,033,628 disclose devices which can pump fluid volumes in the nanoliter or picoliter range, the disclosures of which are hereby incorporated by reference herein in their entireties.

Other pumps suitable for use with microfluidic devices are disclosed in U.S. Pat. No. 10,208,739; and in U.S. Publication Nos. 2015/0050172 and 2017/0167481, the disclosures of which are each hereby incorporated by reference herein in their entireties.

### Control System and Other Modules

The presently disclosed microfluidic devices are communicatively coupled to a control system 104. In some embodiments, the control system 104 is used to send instructions to the various pumps and/or valves so as to regulate a fluid flow (e.g. direction of a fluid and/or reagent flow, a volume of fluid flow, or a flow rate) of any fluids and/or reagents passing through the microfluidic chip. In some embodiments, the control system 104 is configured to send instructions to actuate one or more valves to open or close, including one or more valves disposed in a reservoir, in a conduit, and/or a in channel. In some embodiments, the control system is configured to send instructions to regulate the operation of one or more pumps in fluidic communication with the microfluidic chip, such as to cause the pump to infuse or withdraw fluids, reagents, and/or transfer beads from the processing conduit 105 or any portion thereof.

In some embodiments, the control module 104 may direct a first fluid flow in a first path through a processing conduit 105, e.g. a fluid flow path from an inlet 10, to an inlet channel 12, to a chamber 14, to an outlet channel 13, and to an outlet 11. In other embodiments, the control module 104 may direct a first fluid flow in first and second paths in a processing conduit, where the first and second paths are opposite each other. For instance, through the action of one or more pumps, fluids, reagents, and/or beads may be flow from a first chamber to a second chamber via a transfer channel; and then from the second chamber back to the first chamber.

The control of fluid and/or reagent flow through a microfluid device may be illustrated with reference to FIG. 1C. In some embodiments, a pump 201 may be first commanded by a control system to withdraw a sample, such as a sample provided within a buffer solution, from a sample reservoir 202A. In some embodiments, the buffer may include one or more surfactants. Here, the control system would command valves 207A and 207B to actuate to a position which would allow the sample to flow from the sample reservoir 202A, into the fluid transfer conduit 205B, into the branched conduit 205A, and into the processing conduit 105. In some embodiments, the control system would also command valve 208 to actuate to a position which would permit the flow of fluid into the waste collection vessel 203. As described further herein, molecules within the sample bearing appropriate first reactive functional groups may react with functionalized beads provided within a chamber of the processing conduit 105 and which have corresponding second reactive functional groups. Molecules which are not reacted with the functionalized beads may be flowed, in the buffer solution, through the processing conduit 105, through a pumping conduit 210, through the waste transfer conduit 206, and into the waste collection vessel 203. Finally, the control system would command the valves 207A and 207B to actuate to prevent the sample from flowing from reservoir 202A.

This process may be repeated for one or more additional fluids and/or reagents. For example, a wash buffer stored in reservoir 202B may next be introduced into the processing conduit 105. The control system would command valves 207A and 207B to actuate to a position which would allow a first aliquot of a wash buffer to flow (via a pump 201) from the wash buffer reservoir 202B, into the fluid transfer conduit 205B, into the branched conduit 205A, and into the processing conduit 105. In some embodiments, the control system would also command valve 208 to actuate to a position which would permit the flow of wash buffer into the waste collection vessel 203. In some embodiments, and as described further herein, the wash buffer is flowed through the chamber of the processing conduit so as to remove unbound molecules and/or components introduced in the sample solution stored in reservoir 202A. In some embodiments, the wash is flowed through the processing conduit 105, through a pumping conduit 210, through the waste transfer conduit 206, and into a waste collection vessel 203. Finally, the control system may command the valves 207A and 207B to actuate to prevent additional wash buffer from flowing from reservoir 202B. In some embodiments, additional aliquots of wash buffer may be flowed through the processing conduit 105. For example, two additional aliquots of the same or different wash buffers may be flowed through the processing conduit.

In some embodiments, the above process may be repeated so as to introduce a reagent (e.g. a heated buffer or an enzyme) to release molecules bound to the beads. For instance, the control system would command valves 207A and 207B to actuate to a position which would allow a reagent to flow from reservoir 202D, into the fluid transfer conduit 205B, into the branched conduit 205A, and into the processing conduit 105. In some embodiments, the control system would also command valve 208 to actuate to a position which would permit the flow of fluid into the sample collection vessel 204. As bound molecules are released from the beads, the released molecules are provided to the reagent flowing through the processing conduit 105, and which is ultimately flowed into the sample collection vessel 204.

In some embodiments, the system may further include one or more pressure sensors, temperature sensors and/or flow rate sensors. In some embodiments, the sensors may be coupled to the control system to permit feedback control of the microfluidic system. In some embodiments, the control system is configured to receive data from a sensor (e.g. a flow rate sensor, a temperature sensor, a pressure sensor, a chemical analyzer), process the received data, and regulate fluid a fluid flow, a temperature, a pressure, etc. based on the received and processed data.

In some embodiments, feedback control involves the detection of one or more events or processes occurring in the present microfluidic systems. In some embodiments, detection may involve, for example, determination of at least one characteristic of a fluid, a component within a fluid, interaction between components within regions of the microfluidic chip, within a particular processing conduit 105, or a condition within a region of the microfluidic device or within a portion of a single processing conduit 105 (e.g., temperature, pressure, etc.). By way of example, the control system 104, in some embodiments, is configured to execute a series of instructions to control or operate one or more system components to perform one or more operations, e.g. preprogrammed operations or routines, or to receive feedback from one or more sensor communicatively coupled to the system and command the one or more system components to operate (or cease to operate) depending on the sensor feedback received. In some embodiments, the one or more preprogrammed operations or routines can be performed by one or more programmable processors executing one or more computer programs to perform action, including by operating on received sensor feedback data or imaging data and commanding system components based on that received feedback.

In some embodiments, the microfluidic devices or any component therefore are communicatively coupled to one or more heating modules, cooling modules, and/or mixing modules. In this manner, each processing conduit 105 may be independently heated and/or cooled. In some embodiments, the microfluidic chip, processing conduits, reagent reservoirs, fluid reservoirs, channels, and/or any conduits are each independently in thermal communication with a separate heating and/or cooling module. For instance, each processing conduit 105 may be in thermal communication with a different heating and/or cooling module. In other embodiments, a heating and/or cooling modules are shared between components of the microfluidic devices.

Suitable heating and/or cooling modules include heating blocks, Peltier devices, and/or thermoelectric modules. Suitable Peltier devices include any of those described within U.S. Pat. Nos. 4,685,081, 5,028,988, 5,040,381, and 5,079,618, the disclosures of which are hereby incorporated by reference herein in their entireties. In some embodiments, the control system may be communicatively coupled to the one or more heating and/or cooling modules and configured to command the heating and/or cooling modules to activate to heat and/or cool the microfluidic chip, the processing conduits, the reagent reservoirs, the fluid reservoirs, and/or the conduits to a pre-determined temperature for a pre-determined amount of time. For example, a control module 104 may direct heating from at least one heating module to the microfluidic chip such that a predetermined temperature is reached and/or maintained for a predetermined amount of time. The predetermined temperature may be input to the control system by a user or may be provided within pre-programmed instructions or routines.

In some embodiments, the microfluidic chip or any of the individual processing conduits may be in communication with one or more mixing modules. In some embodiments, the one or more mixing modules include an acoustic wave generator, such as a transducer. In some embodiments, the transducer is a mechanical transducer. In other embodiments, the transducer is a piezoelectric transducer. In some embodiments, the transducer is composed of a piezoelectric wafer that generates a mechanical vibration. In some embodiments, a surface transducer is used to distribute or mix a fluid volume on-slide. Suitable devices and methods for contactless mixing are described in PCT Publication No. WO/2018/215844, the disclosure of which is hereby incorporated by reference.

The control system 104, in some embodiments, includes one or more memories and a programmable processor. To store information, the control system 104 can include, without limitation, one or more storage elements, such as volatile memory, non-volatile memory, read-only memory (ROM), random access memory (RAM), or the like. In some embodiments, the control system 104 is a stand-alone computer, which is external to the system. The storage and/or memory device can be one or more physical apparatuses used to store data or programs on a temporary or permanent basis. In some instances, the device is volatile memory and requires power to maintain stored information. In other instances, the device is non-volatile memory and retains stored information when the digital processing device is not powered. In still other instances, the non-volatile memory comprises flash memory. The non-volatile memory can comprise dynamic random-access memory (DRAM). The non-volatile memory can comprise ferroelectric random access memory (FRAM). The non-volatile memory can comprise phase-change random access memory (PRAM). The device can be a storage device including, by way of non-limiting examples, CD-ROMs, DVDs, flash memory devices, magnetic disk drives, magnetic tapes drives, optical disk drives, and cloud computing based storage.

In some embodiments, the control system 104 is a networked computer which enables control of the system remotely. The term “programmed processor” encompasses all kinds of apparatus, devices, and machines for processing data, including by way of example a programmable microprocessor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

In some embodiments, the system may further include one or more chemical analyzers and/or detectors. In some embodiments, the one or more chemical analyzers may be used to detect cellular components, reagents, byproducts, etc. within a collected fluid stream (e.g. a waste stream). In some embodiments, the chemical analyzers are selected from Qubit for nucleic acid quantification Bioanalyzer for size distribution of NAs, Lightcycler 480 qPCR instrument for nucleic acid quantification, mass spectrometers such as MALDI-TOF MS, LC/MS/MS, CE-MS etc. for molecular identification and/or quantification. Optical microscopy (bright field, fluorescent), spectroscopy such as (IR, NMR, Raman) may also be utilized. In some embodiments, feedback control may be facilitated by in-line coupling any microfluidic device of the present disclosure with an instrument such as CE-MS. In other embodiments, the microfluidic system 100 may be further coupled to a fluorescence microscopy device, such as one included a laser sources and CCD or a CMOS-based imaging sensor and/or camera.

In some embodiments, the microfluidic system 100 may be further coupled to a sequencing device. In some embodiments, the sequencing device is a “next generation sequencing”device.

### Microfluidic Chip Fabrication

The microfluidic chips of the present disclosure may be fabricated according to any method known to those of ordinary skilled in the art. Suitable methods of fabrication include lithography, 3D printing, laser etching, and embossing.

A microfluidic chip may be fabricated of any material suitable for forming a channel and/or conduit. Non-limiting examples of materials include polymers (e.g., polyethylene, polystyrene, polymethylmethacrylate, polycarbonate, poly(dimethylsiloxane), PTFE, PET, and a cyclo-olefin copolymer), glass, quartz, and silicon. The material forming the microfluidic chip and any associated components (e.g., a cover) may be hard or flexible. Those of ordinary skill in the art can readily select suitable material(s) based upon e.g., its rigidity, its inertness to (e.g., freedom from degradation by) a fluid to be passed through it, its robustness at a temperature at which a particular device is to be used, its transparency/opacity to light (e.g., in the ultraviolet and visible regions), and/or the method used to fabricate features in the material. For instance, for injection molded or other extruded articles, the material used may include a thermoplastic (e.g., polypropylene, polycarbonate, acrylonitrile-butadiene-styrene, nylon 6), an elastomer (e.g., polyisoprene, isobutene-isoprene, nitrile, neoprene, ethylene-propylene, hypalon, silicone), a thermoset (e.g., epoxy, unsaturated polyesters, phenolics), or combinations thereof.

The microfluidic chips disclosed herein are typically constructed by single and multilayer soft lithography (MLSL) techniques and/or sacrificial-layer encapsulation methods. The MLSL techniques are particularly useful in some embodiments for producing microfluidic devices which comprise both the control channel and the flow channel. In general, the MLSL technique involves casting a series of elastomeric layers on a micro-machined mold, removing the layers from the mold and then fusing the layers together. In the sacrificial-layer encapsulation approach, patterns of photoresist are deposited wherever a channel is desired. The use of these techniques to fabricate elements of microfluidic devices is described, for example, by Unger et al. (2000) Science 288:113-116; by Chou, et al. (2000) “Integrated Elastomer Fluidic Lab-on-a-chip-Surface Patterning and DNA Diagnostics, in Proceedings of the Solid State Actuator and Sensor Workshop, Hilton Head, S.C.; in PCT Publication WO 01/01025; and in U.S. patent application Ser. No. 09/679,432, filed Oct. 3, 2000, the disclosures of which are incorporated by reference herein in their entireties.

It is believed that MLSL takes advantage of well-established photolithography techniques and advances in microelectronic fabrication technology. The first step in MLSL is to draw a design using computer drafting software, which is then printed on high-resolution masks. Silicon wafers covered in photoresist are exposed to ultraviolet light, which is filtered out in certain regions by the mask. Depending on whether the photoresist is negative or positive, either areas exposed (negative) or not (positive) will crosslink and the resist will polymerize. The unpolymerized resist is soluble in a developer solution and is subsequently washed away. By combining different photoresists and spin coating at different speeds, wafers can be patterned with a variety of different shapes and heights.

In some embodiments, the wafers are then used as molds to transfer the patterns to polydimethylsiloxane (PDMS). In MSL, stacking different layers of PDMS cast from different molds on top of each other is used to create channels in overlapping “flow” and “control” layers. The two (or more) layers are bound together by mixing a potting prepolymer component and a hardener component at complementary stoichiometric ratios to achieve vulcanization. In order to create a simple microfluidic chip, a “thick” layer is cast from the mold containing the flow layer, and the “thin” layer is cast from the mold containing the control layer. After partial vulcanization of both layers, the flow layer is peeled off the mold, and manually aligned to the control layer. These layers are allowed to bond, and then this double slab is peeled from the control mold, and then holes for inlets and outlets are punched and the double slab is bonded to a blank layer of PDMS. After allowing more time to bond, the completed device is mounted on glass slides.

In some embodiments, multiple plates or sheets may be cut (e.g. using a laser cutter) and can be assembled and/or laminated using a double-sided adhesive to create a multi-layer microfluidic device. In some embodiments, the plates or sheets may be plastics, such as Polycarbonate, Acryl, Polypropylene, etc.

### Methods

The present disclosure is also directed to methods of purifying a sample, enriching a sample with desired target molecules, and/or performing solid-phase chemical reactions using the microfluidic devices of the present disclosure. The methods, in some embodiments, employ processing conduits pre-loaded with functionalized beads, such as non-magnetic functionalized beads. In some embodiments, the methods described herein do not require magnetic separation processing or techniques. In some embodiments, the methods are carried out in a closed system which mitigates risk of cross-contamination and/or or sample loss.

### General Method of Purifying a Solution Introduced to a Microfluidic Device

In some embodiments, the present disclosure is directed to methods of purifying a sample using any one of the microfluidic devices 100 described herein.

In some embodiments, a sample may be purified with a microfluidic device 100 of the present disclosure which comprises a processing conduit 105 pre-loaded with beads having a functionalized surface. In some embodiments, the processing conduit may be pre-loaded with between about 10 to about 10,000 functionalized beads. In other embodiments, the processing conduit may be pre-loaded with between about 10 to about 1000 functionalized beads. In yet other embodiments, the processing conduit may be pre-loaded with between about 10 to about 150 functionalized beads. In some embodiments, the pre-loaded functionalized beads are non-magnetic functionalized beads.

In some embodiments, the functionalized surface of the beads includes a first moiety (e.g. a first reactive functional group) which is reactive with a second moiety (e.g. a second reactive functional group) of a molecule (or a conjugate including the molecule) within the sample to be purified. In some embodiments, a “reaction” between a first moiety and a second moiety may mean that a covalent linkage is formed between two reactive groups or two reactive functional groups of the two moieties; or may mean that the two reactive groups or two reactive functional groups of the two moieties associate with each other, interact with each other, hybridize to each other, hydrogen bond with each other, etc. In some embodiments, the “reaction” thus includes binding events, such as the binding of a hapten with an anti-hapten antibody, or the binding of biotin with streptavidin.

In some embodiments, the functionalized surface of the beads introduced to the processing chamber of the microfluidic device may comprise avidin or streptavidin to bind to biotinylated molecules (e.g. molecules conjugated to biotin) within the sample to be purified. By way of another example, in some embodiments thiolated molecules may be bound to gold surfaces. By way of yet another example, amine-terminated molecules may be bound to an NHS-activated bead surface.

In some embodiments, the functionalized surface of the beads comprises immobilized antibodies, which may be used to bind to molecules including or conjugated to specific antigenic molecules. In yet other embodiments, the functionalized surface of the beads comprises enzymes, which may be used to bind to molecules including or conjugated to specific enzyme substrates. In further embodiments, the functionalized surface of the beads comprises receptors, which may be used to bind to molecules including or conjugated to specific receptor ligands. In yet further embodiments, the functionalized surface of the beads comprises lectins, which may be used to bind to molecules including or conjugated to specific polysaccharides. In even further embodiments, the functionalized surface of the beads comprises nucleic acids, which may be used to bind to molecules including or conjugated to complementary base sequences. In some embodiments, DNA/RNA aptamers tethered onto the bead surface may specifically bind to its target analytes such as small molecules, peptides, proteins, cells.

In some embodiments, and regardless as to how the surface of the beads are functionalized, the beads may themselves be non-magnetic. Suitable non-magnetic beads are described in U.S. Pat. No. 5,328,603, the disclosure of which is hereby incorporated by reference herein in its entirety.

In general, the methods of purifying a sample using the microfluidic devices of the present disclosure comprise (i) binding a subset of appropriately functionalized molecules within an input sample to be purified to functionalized beads present within a chamber of a processing conduit; (ii) flowing one or more wash solutions through the processing conduit to remove unbound molecules, reagents, and/or impurities included within the input sample; and (iii) flowing a solution through the processing conduit to release the bound molecules from the functionalized beads. In some embodiments, one or more reagents may be optionally introduced into the processing conduits to derivatize the subset of molecules bound to the functionalized beads. In some embodiments, the methods do not rely on magnetic beads or magnetic separation for purification. Rather, purification is effectuated by flowing a series of fluids and/or reagents through the processing conduit.

In some embodiments, the subset of appropriately functionalized molecules are bound to the functionalized beads by introducing the input sample to the processing conduit pre-loaded with functionalized beads, where the input sample includes the subset of appropriately functionalized molecules. In some embodiments, the subset of appropriately functionalized molecules within the input sample include a first moiety which is capable of reacting with a second moiety of the functionalized beads. In some embodiments, the subset of appropriately functionalized molecules are generated prior to the input sample's introduction to the processing conduit. In some embodiments, the subset of appropriately functionalized molecules are generated by contacting the input sample with a reagent that selectively reacts with a subset of the molecules within the input sample. By way of example, the reagent may be an oligonucleotide sequence having a moiety capable of reacting with a functionalized bead. In some embodiments, the reaction is a conjugation reaction where a moiety capable of reacting with a functionalized bead is introduced to the subset of molecules. In some embodiments, the introduced moiety is a first moiety which is capable of reacting with the second moiety of the functionalized beads.

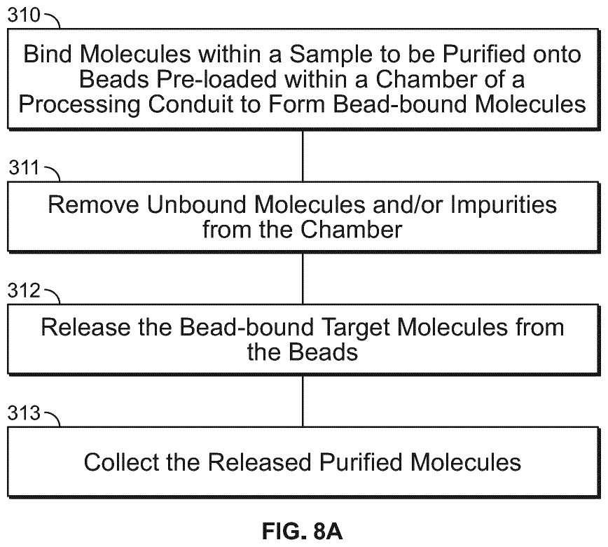

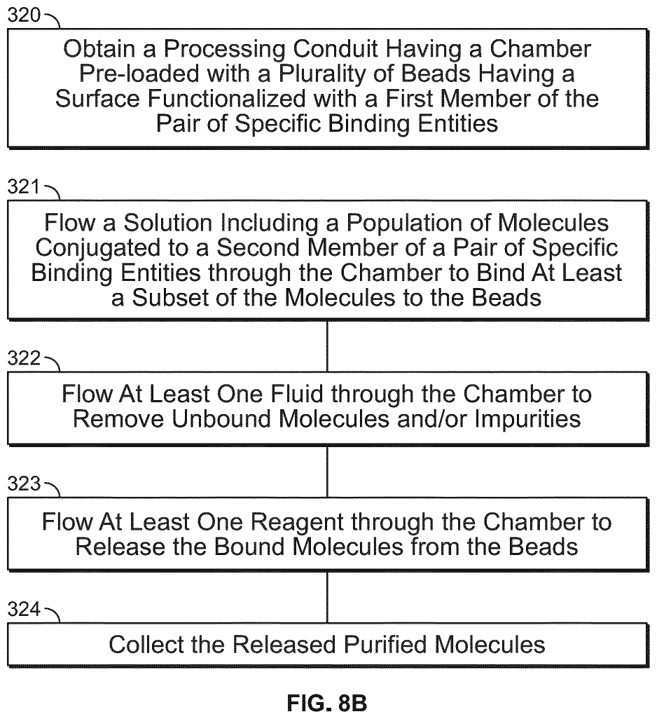

Methods of purifying an input sample are illustrated in FIGS. 8A and 8B. In some embodiments, a processing conduit having a chamber pre-loaded with a plurality of beads having a functionalized surface is first obtained (step 320). In some embodiments, the chamber is pre-loaded with a plurality of non-magnetic beads having a functionalized surface. In some embodiments, the functionalized surface includes a first member of a pair of specific binding entities. In some embodiments, the functionalized surface includes a first moiety selected from avidin, streptavidin, and antibody, an enzyme, a receptor, a lectin, a nucleic acid sequence, etc. In some embodiments, the functionalized beads may be introduced by pumping the beads from a bead storage vessel into one or more ducts in fluidic communication with the chamber. In some embodiments, the chamber is sealed after the beads are introduced.

In some embodiments, an input sample including a subset of molecules to be purified is then introduced to the processing conduit having the chamber pre-loaded with the plurality of beads such that bead-molecule complexes may be formed (step 310). In some embodiments, the bead-molecule complexes are formed by flowing the input sample including the subset of the molecules to be purified into and through the chamber of the processing conduit (step 321). For instance, the input sample may be provided in a fluid or a buffer solution and introduced by pumping the fluid or buffer solution into an inlet of a processing conduit, flowing the fluid or buffer solution through an inlet channel of the processing conduit and into the chamber having the pre-loaded beads.

In some embodiments, the input sample comprises a subset of molecules having a second moiety capable of reacting with the first moiety of the functionalized beads such that the first and second moieties may react with one another. In some embodiments, the subset of molecules having the second moiety are generated by contacting the input sample with a reagent that selectively reacts with the subset of the molecules within the input sample.

In some embodiments, the input sample is flowed through the processing conduit at a rate which permits the molecules having the second moiety time to react with the functionalized beads. For example, in some embodiments, the sample may be flowed through the processing conduit at a rate of between about 0.1 mL per minute to about 1000 mL per minute. In other embodiments, the sample may be flowed through the processing conduit at a rate of between about 0.1 mL per minute to about 100 mL per minute.

In some embodiments, the subset molecules having the second moiety and to be purified are allowed time to incubate with the functionalized beads. In some embodiments, an incubation period may range from between about 15 seconds to about 90 minutes. In some embodiments, an incubation period may range from between about 1 minute to about 60 minutes. In other embodiments, an incubation period may range from between about 1 minute to about 20 minutes. In those embodiments requiring an incubation time, once the input sample is flowed into the chamber of the processing conduit, the one or more pumps fluidically coupled to the processing conduit may be commanded to turn off (or slow the rate of fluid flow) for the predetermined incubation time.

Following the binding of the subset of molecules to be purified to the beads (i.e. following the formation of the bead-molecule complexes), in some embodiments, one or more fluids are then flowed through the processing conduit (step 322) to remove unbound molecules and/or impurities from the chamber of the processing conduit (step 311). For instance, a fluid (e.g. a buffer solution) may introduced by pumping the fluid into an inlet of a processing conduit, flowing the fluid through an inlet channel of the processing conduit and into the chamber of the processing conduit. In some embodiments, fluid (e.g. a first type of buffer) is flowed through the chamber once (e.g. a predetermined volume of a single type of buffer is flowed through the chamber once). In other embodiments, the same or different fluids are flowed through the chamber two or more times (e.g. a predetermined first volume of a first fluid is flowed through the chamber, and then a predetermined volume of a second fluid is flowed through the chamber). In yet other embodiments, different fluids, e.g. different buffers, are sequentially flowed through chamber three or more times.

In some embodiments, the fluid (e.g. buffer) flowed into the chamber is held within the chamber for a predetermined time, e.g. a time period ranging from between about 1 minutes to about 60 minutes. In other embodiments, the fluid introduced into the processing conduit is agitated, such as by introducing vibrations into the processing conduit (e.g. through a transducer in communication with the processing conduit) or by directing one or more pumps to repeatedly infuse and withdraw small quantities of the fluid from the processing conduit.

In some embodiments, a fluid waste stream flowing out of the chamber, through the outlet channel, and through the outlet is monitored, such as with camera, e.g. a fluorescent camera, to determine if substantially all unbound molecules and/or impurities have been removed. In some embodiments, a fluorescent camera with laser source can be utilized at the outlet to monitor the fluorescent signals emitted from unbound molecules and/or impurities, and this signal can be fed back to the control system to command the operation of the valves and/or pumps. In other embodiments, a conductivity detector including two metal wires may be also used to detect local pH changes caused by a molecular composition locally and again this acquired pH data may be used for feedback control.

For instance, as fluid is flowed through the chamber, into an outlet conduit, and through an outlet, a detector (e.g. a fluorescent detector) in communication therewith may be used to detect and/or quantify the unbound molecules and/or impurities within the waste stream. The fluorescent detector in communication with the outlet of the processing conduit may also be used to determine if target molecules are being lost and, if so, processing parameters may be adjusted to mitigate such loss. In some embodiments, fluid is repeatedly and/or sequentially introduced until substantially all of the unbound molecules and/or impurities have been removed from the chamber as determined by fluorescent detector. In other embodiments, fluid is repeatedly and/or sequentially introduced until a quantity of the unbound molecules and/or one or more impurities in the waste stream is less than a predetermined impurity threshold value.

Following the removal of substantially all unbound molecules and/or impurities from the chamber of the processing conduit, the subset of molecules bound to the beads are released from the beads (step 312) and subsequently collected (steps 313 and 324). In some embodiments, the subset of molecules are released by flowing a fluid or reagent into the processing conduit suitable for releasing the molecule from the bead (step 323). In some embodiments, the fluid is a buffer fluid that is pre-heated or heated in-situ to a predetermined temperature. In some embodiments, the fluid is heated to a temperature ranging from between about 85° C. to about 105° C. In other embodiments, the fluid is heated to a temperature ranging from between about 90° C. to about 100° C. In some embodiments, pre-heated fluid may be heated by commanding one or more heating modules in thermal communication with a reservoir to head the fluid to a predetermined temperature. In some embodiments, heaters in thermal communication with the processing conduit may be commanded to heat an introduced fluid to a predetermined temperature. In some embodiments, a reagent is introduced to effectuate release of the subset of molecules. In some embodiments, the reagent is an enzyme, e.g. an enzyme capable of cleaving a molecule at a predetermined location or at a specific bond. In some embodiments, the released subset of molecules may then be used in one or more downstream processes, e.g. further chemical reactions, sequencing, etc.

### Target Enrichment Using a Microfluidic Device

The present disclosure also relates to a method of reducing the complexity of a nucleic acid sample by enriching for specific nucleic acid target sequences in the nucleic acid sample. In some embodiments, the present disclosure is directed to methods of enriching for specific target sequences in a nucleic acid sample using libraries of oligonucleotide probes. The nucleic acid sample enriched for the specific target sequences may then be used in downstream sequencing operations. In some embodiments, the methods of target enrichment described herein do not utilize magnetic beads or magnetic separation techniques.

In some embodiments, the present disclosure is directed to methods of target enrichment using any one of the microfluidic devices described herein. The present disclosure is also directed to methods of sequencing using a target enriched sample, such as a target enriched sample prepared using any one of the microfluidic devices described herein. In some embodiments, targeted sequencing, in general, enables the detection of known and novel variants in selected sets of genes or genomic regions. In some embodiments, the target enriched sample is sequencing using next-generation sequencing. For example, when a sample solution including nucleic acid sequences of interest are flowed through a chamber of a processing conduit pre-loaded with a plurality of functionalized beads, nucleic acids of interest are bound onto the bead surface through various chemistries. In some embodiments, buffers (e.g. wash buffers) are subsequently flowed through the processing conduit and around the beads disposed therein to remove unbound non-target nucleic acids and impurities. In some embodiments, an eluant is then introduced into the chamber to release target nucleic acids via temperature change or enzymatic cleavage. In some embodiments, the released nucleic acids are then collected through the outlet and transferred to downstream processes.

In some embodiments, target enrichment includes obtaining a genomic sample. In some embodiments, the obtained genomic sample is a sample derived from a mammalian subject, e.g. a human subject. In some embodiments, the obtained genomic sample is a blood sample or a blood plasma sample obtained from a mammalian subject, e.g. a blood sample or a blood plasma sample obtained from a human subject. In some embodiments, the obtained genomic sample is in the form of cell-free nucleic acids. In some embodiments, the obtained genomic sample in the form of cell-free nucleic acids comprises DNA and/or RNA. In some embodiments, the cell-free DNA typically ranges in size from between about 200 bp to about 130 bp. In some embodiments, the cell-free DNA typically ranges in size from between about 190 bp to about 140 bp. In some embodiments, the cell-free DNA typically ranges in size from between about 180 bp to about 150 bp. Non-limiting examples of cell-free nucleic acids include circulating tumor DNA (ctDNA) and fetal cell-free DNA present in maternal blood and blood plasma. In some embodiments, the present disclosure also encompasses isolation of various types of cell-free RNA.

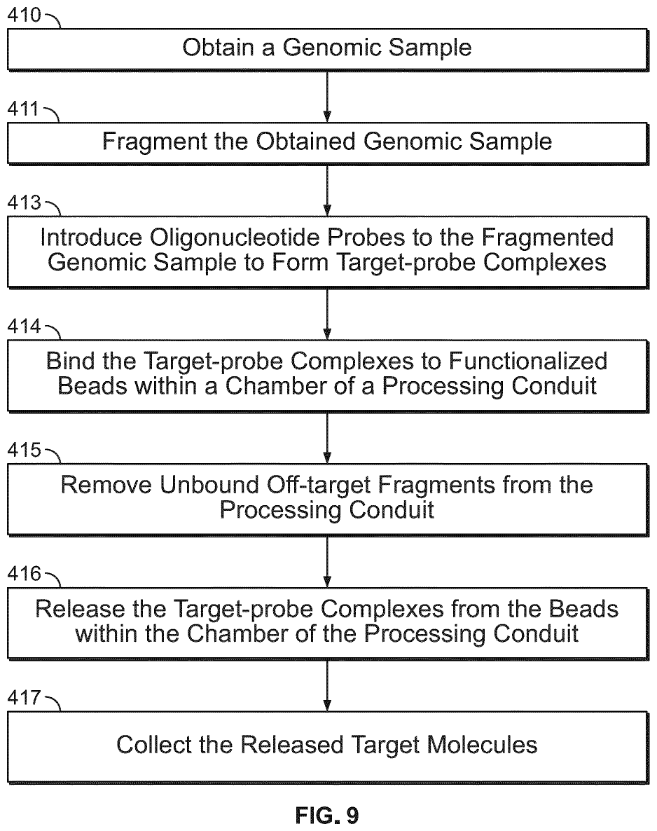

Alternatively, and with reference to FIG. 9, in some embodiments, target enrichment includes obtaining a genomic sample, e.g. a genomic DNA sample acquired from a human patient (step 410). In some embodiments, the obtained genomic sample is sheared into fragments to provide a population of nucleic acid fragments (step 411). In some embodiments, shearing of the obtained genomic sample is effectuated using mechanical (e.g. nebulization or sonication) and/or enzymatic fragmentation (e.g. restriction endonucleases).

In some embodiments, the generated nucleic acid fragments are randomly sized. In some embodiments, the generated nucleic acid fragments have a length which are less than about 1000 base pairs. In other embodiments, the generated nucleic acid fragments comprises sequence fragments having a sequence size ranging from between about 100 to about 1000 base pairs in length. In yet other embodiments, the generated nucleic acid fragments comprises sequence fragments having a sequence size ranging from between about 500 to about 750 base pairs in length. In some embodiments, adapters, such as those including a specific barcode sequence, are then added via a ligation reaction to the population of nucleic acid.

Following the obtaining of the genomic sample (and/or the optional fragmentation of the obtained genomic sample), in some embodiments a pool of oligonucleotide probes, such as oligonucleotide probes conjugated to a first member of a pair of specific binding entities, are introduced to the obtained genomic sample or the population of nucleic acid fragments. In some embodiments, the pool of oligonucleotide probes are introduced to a buffer solution including the obtained genomic sample or the population of nucleic acid fragments (step 413). In some embodiments, the oligonucleotide probes are reference populations of nucleic acid sequences capable of hybridizing to complementary nucleic acid sequences within the genomic sample or the population nucleic acid fragments. In some embodiments, the oligonucleotide probes are designed to target desired genes, exons, and/or other genomic regions of interest within the genomic sample or the population of nucleic acid fragments. In some embodiments, the oligonucleotide probes are selected such that the oligonucleotide probes relate to, by way of non-limiting examples, a set of genes of interest, all of the exons of a genome, particular genetic regions of interest, disease or physiological states and the like.

In some embodiments, the oligonucleotide probes are DNA capture probes. In some embodiments, the DNA capture probes include a pool of Roche SeqCap EZ Probes (available from Roche Sequencing and Life Sciences, Indianapolis, IND). In some embodiments, a pool of Roche SeqCap EZ Probes include a mixture of different biotinylated single-stranded DNA oligonucleotides in solution, each with a specific sequence, where the length of individual oligonucleotides can range from about 50 nucleotides to about 100 nucleotides with a typical size of about 75 nucleotides. In some embodiments, a Roche SeqCap EZ Probe Pool can be used in sequence capture experiments to hybridize to targeted complementary fragments of a DNA sequencing library and thus to capture and enrich them relative to untargeted fragments of the same DNA sequencing library prior to sequencing. The DNA sequencing library may be constructed from genomic DNA for genome analysis, or from cDNA prepared from RNA or mRNA for transcriptome analysis, and it may be constructed from the DNA or cDNA of any species of organism from which these nucleic acids can be extracted.

In some embodiments, the oligonucleotide probes hybridize to a first subset of complementary nucleic acids within the genomic sample or nucleic acid fragments within the population of nucleic acid fragments which include the desired genes, exons, and/or other genomic regions of interest to form target-probe complexes having a first member of a pair of specific binding entities. In some embodiments, a second subset of nucleic acids or nucleic acid fragments within the obtained genomic sample or the solution of nucleic acid fragments, respectively, that do not include the desired genes, exons, and/or other genomic regions of interest do not form target-probe complexes and are referred to as “off-target nucleic acids” or “off-target fragments.” As such, following the introduction of the oligonucleotide probes, any solution for enrichment may include formed target-probe complexes, off-target nucleic acids or off-target fragments, and/or free probes (assuming that an excess amount of oligonucleotide probes are provided to any solution including adapter-ligated DNA fragments). In some embodiments, the solution for enrichment is provided in a buffer solution.

Subsequently, the solution for enrichment, including the formed target-probe complexes, off-target nucleic acids and/or off-target fragments, are introduced to a chamber of a processing conduit of a microfluidic device, where the chamber is pre-loaded with a plurality of beads. In some embodiments, any of the microfluidic devices described herein may be utilized for target enrichment. In some embodiments, the microfluidic device includes a processing conduit having no moving parts, e.g. no moving mechanical parts. In some embodiments, the plurality of beads pre-loaded into the chamber are non-magnetic and wherein the processing conduit of the microfluidic device includes no magnetic strips. In some embodiments, the microfluidic device utilized for target enrichment does not rely on magnetic separation.

In some embodiments, the processing conduit may be pre-loaded with between about 10 to about 10,000 functionalized beads or more. In other embodiments, the processing conduit may be pre-load with between about 10 to about 1000 functionalized beads. In yet other embodiments, the processing conduit may be pre-load with between about 10 to about 150 functionalized beads. In some embodiments, the plurality of beads are functionalized with a plurality of second members of the pair of specific binding entities. In some embodiments, the second members of the pair of specific binding entities comprises avidin or streptavidin.

In some embodiments, the solution for enrichment is flowed into an inlet of a processing conduit, through an inlet conduit of the processing conduit, and into the chamber pre-loaded with the plurality of functionalized beads. In some embodiments, the first members of the pair of specific binding entities of the target-probe complexes react with the second members of the pair of specific binding entities of the functionalized beads such that the target-probe complexes within the solution for enrichment become bound to the beads within the chamber of the processing conduit (step 414). Likewise, in some embodiments the first members of the pair of specific binding entities of any free probes in the solution for enrichment become bound to the functionalized beads. In this manner, the target-probe complexes and/or free probes become bound to the beads within the chamber. As such, the beads within the chamber include immobilized (i.e. bead bound) target-probe complex and free probes. Also included within the reaction chamber, in some embodiments, are unbound, off-target nucleic acids or off-target fragments.

In some embodiments, the target-probe complexes are allowed time to incubate with the functionalized beads. In some embodiments, an incubation period may range from between about 1 minute to about 60 minutes. In other embodiments, an incubation period may range from between about 1 minute to about 40 minutes. In yet other embodiments, an incubation period may range from between about 1 minute to about 20 minutes. In those embodiments where an incubation time is utilized, once the input sample is flowed into the chamber of the processing conduit, the one or more pumps fluidically coupled to the processing conduit may be commanded to turn off for the predetermined incubation time.

Following the binding of the target-probe complexes to the functionalized beads and/or the binding of free-probes to the beads, unbound off-target nucleic acids, off-target fragments, reagents, and/or impurities are then removed from the chamber of the processing conduit (step 415). In some embodiments, removal of the off-target fragments that were not complementary to any the oligonucleotide probes introduced to the solution for enrichment enriches the remaining immobilized target genomic material.

For example, in some embodiments, one or more fluids are flowed through the processing conduit to remove the off-target nucleic acids or off-target fragments, reagents, and/or impurities from the chamber of the processing conduit. In some embodiments, a fluid (e.g. a buffer solution) may introduced by pumping the fluid into an inlet of a processing conduit, flowing the fluid through an inlet channel of the processing conduit and into the chamber of the processing conduit. In some embodiments, fluid (e.g. a first type of buffer) is flowed through the chamber once (e.g. a predetermined volume of the single type of buffer is flowed through the chamber once). In other embodiments, the same or different fluids are flowed through the chamber two or more times (e.g. a predetermined first volume of a first fluid is flowed through the chamber, and then a predetermined volume of a second fluid is flowed through the chamber). In yet other embodiments, different fluids, e.g. different buffers, are sequentially flowed through chamber three or more times.

In some embodiments, the beads disposed within the chamber of the processing conduit are sequentially washed three or more times. In some embodiments, the beads disposed within the chamber of the processing conduit are sequentially washed three of more times with a buffer having a pH ranging from between about 1 to about 14. In other embodiments, the beads disposed within the chamber of the processing conduit are washed three of more times with a buffer having a pH ranging from between about 3 to about 12. In yet other embodiments, the beads disposed within the chamber of the processing conduit are washed three of more times with a buffer having a pH ranging from between about 5 to about 8. In some embodiments, the beads are sequentially washed with phosphate buffered saline.

In some embodiments, the fluid (e.g. buffer) flowed into the chamber is held within the chamber for a predetermined time, e.g. a time period ranging from between about 1 minute to about 60 minutes. In other embodiments, the fluid introduced into the processing conduit is agitated, such as by introducing vibrations into the processing conduit (e.g. through a transducer in communication with the processing conduit) or by directing one or more pumps to repeatedly infuse and withdraw small quantities of the fluid from the processing conduit.

Following the removal of substantially all off-target nucleic acids, off-target fragments, reagents, and/or impurities from the chamber of the processing conduit, the target molecules are removed from the chamber (step 416) (i.e. released from the beads) and subsequently collected (step 417). In some embodiments, the target molecules or target molecule complexes are released by flowing a fluid or reagent into the processing conduit suitable for releasing the target molecule or the target molecule complex from the particle or bead. In some embodiments, the target molecules are removed from the chamber by flowing a heated fluid through the processing conduit.

For example, a pre-heated fluid may be introduced to the chamber to effectuate release. In some embodiments, the temperature-of the pre-heated fluid may range from between about 4° C. to about 150° C. In other embodiments, the temperature-of the pre-heated fluid may range from between about 20° C. to about 95° C. In yet other embodiments, the temperature-of the pre-heated fluid may range from between about 37° C. to about 65° C. In some embodiments, the heated fluid permits the denaturation of the target-probe complexes. In some embodiments, the fluid is a heated buffer. Non-liming examples of buffers include citric acid, potassium dihydrogen phosphate, boric acid, diethyl barbituric acid, piperazine-N,N′-bis(2-ethanesulfonic acid), dimethylarsinic acid, 2-(N-morpholino)ethanesulfonic acid, tris(hydroxymethyl)methylamine (TRIS), 2-(N-morpholino)ethanesulfonic acid (TAPS), N,N-bis(2-hydroxyethyl)glycine(Bicine), N-tris(hydroxymethyl)methylglycine (Tricine), 4-2-hydroxyethyl-1-piperazineethanesulfonic acid (HEPES), 2-{[tris(hydroxymethyl)methyl]amino} ethanesulfonic acid (TES), and combinations thereof. In some embodiments, the unmasking agent is water. In other embodiments, the buffer solution may be comprised of tris(hydroxymethyl)methylamine (TRIS), 2-(N-morpholino)ethanesulfonic acid (TAPS), N,N-bis(2-hydroxyethyl)glycine(Bicine), N-tris(hydroxymethyl)methylglycine (Tricine), 4-2-hydroxyethyl-1-piperazineethanesulfonic acid (HEPES), 2-{[tris(hydroxymethyl)methyl]amino}ethanesulfonic acid (TES), or a combination thereof. In some embodiments, the buffer has a pH ranging from about 5 to about 9.

In some embodiments, a fluid is introduced into the chamber and then heated. In some embodiments, the fluids, beads, and/or processing conduit are heated to a temperature ranging from between about 85° C. to about 105° C. In other embodiments, the fluids, beads, and/or processing conduit are heated to a temperature ranging from between about 90°°C. to about 100° C. In yet other embodiments, the fluids, beads, and/or processing conduit are heated to a temperature ranging from between about 85° C. to about 105° C.

In other embodiments, a reagent (e.g. an enzyme) is introduced to effectuate release. Examples of suitable enzymes include trypsin (which cleaves the peptide bonds at the carboxyl end of lysine and arginine residues) and clostripain (which cleaves at the carboxyl side of arginine residues).

In some embodiments, the reagent is allowed time to incubate with the bead-bound target-probe complexes and/or the bead-bound free probes. In some embodiments, an incubation period may range from between about 1 minute to about 60 minutes. In other embodiments, an incubation period may range from between about 1 minute to about 40 minutes. In yet other embodiments, an incubation period may range from between about 1 minute to about 20 minutes.

The released target molecules may then be used in one or more downstream processes, e.g. sequencing, amplification, further coupling, etc. In some embodiments, sequencing may be performed according to any method known to those of ordinary skill in the art. In some embodiments, sequencing methods include Sanger sequencing and dye-terminator sequencing, as well as next-generation sequencing technologies such as pyrosequencing, nanopore sequencing, micropore-based sequencing, nanoball sequencing, MPSS, SOLID, Illumina, Ion Torrent, Starlite, SMRT, tSMS, sequencing by synthesis, sequencing by ligation, mass spectrometry sequencing, polymerase sequencing, RNA polymerase (RNAP) sequencing, microscopy-based sequencing, microfluidic Sanger sequencing, microscopy-based sequencing, RNAP sequencing, etc. Instruments and methods of sequencing are disclosed, for example, in PCT Publication Nos. WO2014144478, WO2015058093, WO2014106076 and WO2013068528, the disclosures of which are hereby incorporated by reference in their entireties.

The method of target enrichment and the flow of fluids and/or reagents through a microfluidic device may be illustrated with reference to FIG. 1E. In some embodiments, a solution to be enriched including target-probe complexes, off-target nucleic acids, off-target fragments, and/or free probes is stored, for example, in reservoir 264. In some embodiments, the solution to be enriched includes a buffer. A control system may then send signals to a valves 257 and 255 to open to permit the withdrawal of the solution to be enriched by one or both of pumps 250A and 250B. The control may then send further signals to valves 257 and 255 to actuate such that the withdrawn solution for enrichment may then be infused into the processing conduit 105, where the processing conduit is pre-loaded with functionalized beads. In some embodiments, the pre-loaded beads are non-magnetic. In some embodiments, the processing conduit 105 includes no moving parts. As noted above, the target-probe complexes and free-probes may become bound to the functionalized beads.

Subsequently, the control system may send signals to valves 255 and 258 to permit the withdrawal of a first buffer from reservoir 260 and into one or both of the pumps 250A and 250B. The control system may then command valves 255, 258, and 257 to actuate such that the first buffer is infused into the processing conduit 105 such that off-target nucleic acids or off-target fragments are removed from the chamber. These steps may be repeated one or more times such that the same first buffer is withdrawn from reservoir 260 and infused into the processing conduit 105.

Next, the control system may send signals to valves 255 and 256 to permit the withdrawal of a second buffer from reservoir 263 and into one or both of the pumps 250A and 250B. The control system may then command valves 255, 256, and 257 to actuate such that the second buffer is infused into the processing conduit 105 such that off-target nucleic acids or off-target fragments are removed from the chamber. These steps may be repeated one or more times such that the same second buffer is withdrawn from reservoir 263 and infused into the processing conduit 105.

The control system may then send signals to valves 255 and 256 to permit the withdrawal of a third buffer from reservoir 262 and into one or both of the pumps 250A and 250B. The control system may then command valves 255, 256, and 257 to actuate such that the third buffer is infused into the processing conduit 105 such that off-target nucleic acids or off-target fragments are removed from the chamber. These steps may be repeated one or more times such that the same third buffer is withdrawn from reservoir 262 and infused into the processing conduit 105.

Finally, the control system may send signals to valves 255 and 258 to permit the withdrawal of a reagent from reservoir 261 and into one or both of the pumps 250A and 250B. The control system may then command valves 255, 258, and 257 to actuate such that the reagent is infused into the processing conduit 105 such that the bound target-probe complexes and bound free-probes are released from the beads. In embodiments where the reagent is a buffer, the control system may command heaters in thermal communication with the processing conduit 105 to head the introduced reagent. The eluent including the released target-probe complexes and the release free-probes may then be collected and used in downstream processing, e.g. next-generation sequencing.

### Reactions/Solid-State Synthesis Carried out Within a Processing Conduit of a Microfluidic Device

The present disclosure provides, in some embodiments, a method of performing one or more solid-phase reactions in a processing conduit of a microfluidic device. In general, the methods of performing one or more solid-phase reactions in a processing conduit of a microfluidic device of the present disclosure comprise (i) binding a subset of appropriately functionalized molecules within an input sample to functionalized beads present within a chamber of a processing conduit; (ii) flowing one or more wash solutions through the processing conduit to remove unbound molecules, reagents, and/or impurities included within the input sample; (iii) flowing one or more reagents into the processing conduit; and (iv) flowing a solution through the processing conduit to release the bound molecules from the functionalized beads. In some embodiments, one or more reagents may be optionally introduced into the processing conduits to derivatize the subset of molecules bound to the functionalized beads.

In some embodiments, the subset of appropriately functionalized molecules are bound to the functionalized beads by introducing the input sample including the subset of appropriately functionalized molecules to the processing conduit pre-loaded with functionalized beads. In some embodiments, the subset of appropriately functionalized molecules within the input sample include a first moiety which is capable of reacting with a second moiety of the functionalized beads. In some embodiments, the subset of appropriately functionalized molecules are generated prior to the input sample's introduction to the processing conduit. In some embodiments, an input sample including a subset of molecules to be further reacted is then introduced to the processing conduit having the chamber pre-loaded with the plurality of beads such that bead-molecule complexes may be formed. In some embodiments, the bead-molecule complexes are formed by flowing the input sample including the subset of the molecules to be further reacted into and through the chamber of the processing conduit.

Following the binding of the subset of molecules to be further reacted to the beads (i.e. following the formation of the bead-molecule complexes), in some embodiments, one or more fluids are then flowed through the processing conduit to remove unbound molecules and/or impurities from the chamber of the processing conduit. This step of removing unbound molecules and/or impurities may be repeated one or more times, e.g. two or more times, three or more times, four or more times, etc.

Subsequently, one or more reagents may be flowed into the processing conduit. In some embodiments, the one or more reagents are reactive with the molecules bound to the beads. For example, the molecules bound to the beads may be an oligonucleotide and the reagents may include nucleotides or short oligonucleotides for conjugation. By way of another example, the molecules bound to the beads may be peptides and the reagents may include amino acids or short peptides for conjugation. In some embodiments, the molecules bound to the beads may be DNA or RNA aptamers; and the reagents may include small molecules, peptides, proteins or cells which specifically bind to surface-immobilized aptamer molecules. Following a first reaction with a first introduced reagent, a fluid may be introduced into the processing conduit to remove excess reagents and/or any impurities. This washing step may be performed one or more times. The process of introducing one or more reagents and/or introducing one or more fluids to remove excess reagents and/or impurities may be repeated one or more times, e.g. two or more times, three or more times, four or more times, etc.

After all desired reactions have been carried out, the subset of molecules bound to the beads are then released from the beads and subsequently collected. In some embodiments, the subset of molecules are released by flowing a fluid or reagent into the processing conduit suitable for releasing the molecule from the bead. In some embodiments, the fluid is a buffer fluid that is pre-heated or heated in-situ to a predetermined temperature. In some embodiments, the fluid is heated to a temperature ranging from between about 85° C. to about 105° C. In other embodiments, the fluid is heated to a temperature ranging from between about 90° C. to about 100° C. In some embodiments, a reagent is introduced to effectuate release of the subset of molecules. In some embodiments, the reagent is an enzyme. In some embodiments, the released subset of molecules may then be used in one or more downstream processes.

### EXAMPLES

### Example 1—Capture of Biotinylated Oligonucleotides

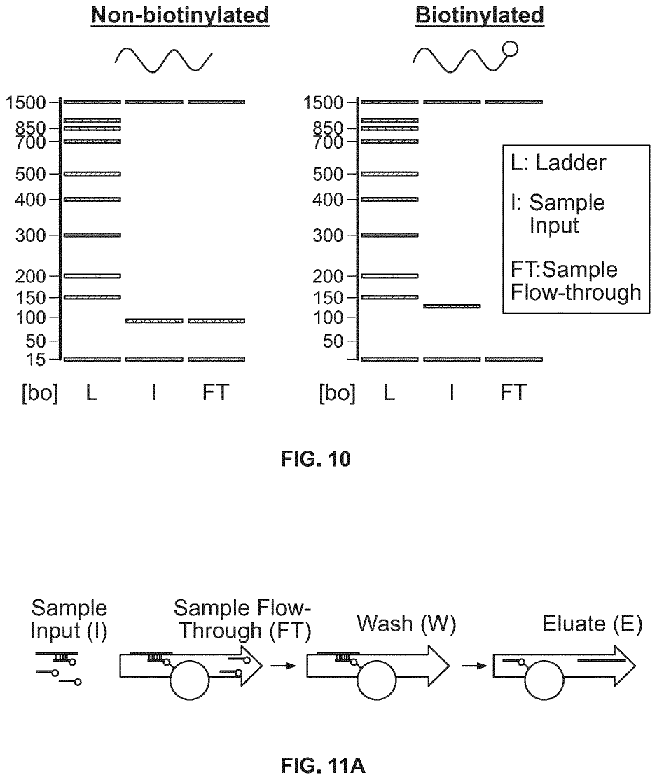

To test the target capture in the device, two microfluidic bead trapping devices packed with streptavidin-functionalized beads (Streptavidin Plus UltraLink Resin from Pierce), were prepared. A 50 μL sample input with biotinylated oligonucleotides was flowed through one of the microfluidic devices, and the flow-through eluent was collected for analysis. As a control experiment, non-biotinylated oligonucleotides were processed through the second microfluidic device in parallel. The flow-through (Ff) and the sample input (I) prior to processing were then analyzed using a Bioanalyzer DNA1000 kit. In the case of the biotinylated oligonucleotide sample, oligonucleotides were captured on the beads via a streptavidin-biotin interaction, and therefore the flow-through did not present any detectable peaks representing the oligo (right panel in FIG. 10). The non-biotinylated oligonucleotides were not bound to the bead surfaces, but passed through the bead-packed chamber, which resulted in comparable electropherograms between sample input and flow-through (left-panel in FIG. 10). This demonstrated that highly efficient capture of the biotinylated oligonucleotides with microfluidic devices of the present disclosure could be achieved.

### Example 2—Capture and Temperature-Mediated Release of Target

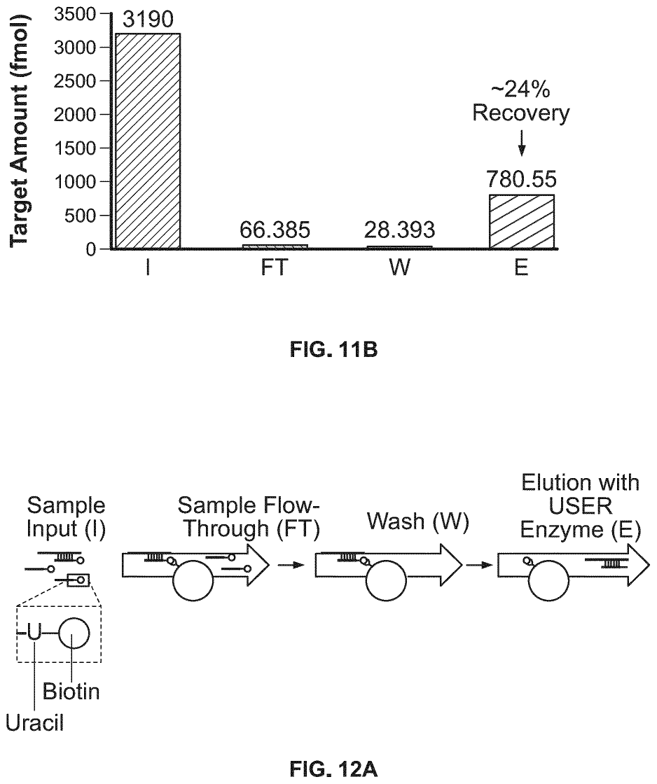

The capture of target-probe complexes followed by temperature-mediated release of the target oligonucleotides was tested. Target oligonucleotides were first annealed with biotinylated probes containing complementary sequences. A 10-fold excess amount of the probe was used in order to ensure all targets were complexed with the biotinylated probes. A 50 μL of sample input comprising target-probe complexes (500 pmol) was then flowed through the microfluidic device packed with streptavidin-coated beads. A total of 5 fractions (10 μL each×5 times) of flow-through (Ff) was collected. The microfluidic device was then washed with PBS buffer and the wash buffer was collected (W). Finally, the bead-packed chamber of the microfluidic device was heated to about 95° C. to denature the target oligos from the probe attached to the bead surface, and the eluate was collected (E). A qPCR was subsequently run on 1 sample input (I) and the 3 collections from the device (Ff, W, E) using a primer specific to the target oligo. The results are illustrated in FIG. 11, which demonstrate that target-probe complexes were captured by the beads within the microfluidic device with a capture efficiency of 98 %, and with minimal loss by washing (<1%). Total recovery after temperature-mediated release was calculated to be about 24%. Such a low releasing efficiency was attributed to the inefficient liquid collection, which was observed to be impeded by bubbles generated in the bead-packed chamber at 95° C.

### Example 3—Capture and Enzymatic Release of Target

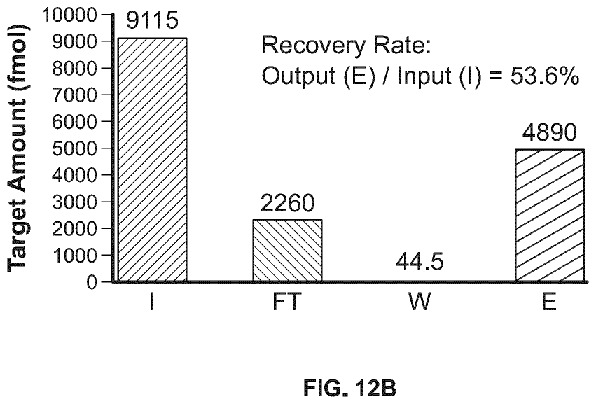

The capture and enzymatic release of target-probe complexes was next tested. For the specific enzymatic cleavage, uracil was placed between probe sequence and biotin (FIG. 12). Target oligonucleotides were then annealed with the uracil-incorporated biotinylated probes. A 10-fold excess amount of the probe was used in order to ensure all targets were complexed with the biotinylated probes. A 50 μL of sample input containing target-probe complexes (500 pmol) was then flowed through the microfluidic device packed with streptavidin-coated beads. A total 5 fractions (10 μL each×5 times) of flow-through (Ff) was collected. The device was then washed with PBS buffer and the wash buffer was collected (W). Finally, the bead-packed chamber was incubated with Uracil-Specific Excision Reagent (“USER”) enzyme to cleave the uracil site and release the target-probe complexes from the bead surface, and the eluate was collected (E). Here, the uracil is located between the probe sequence and biotin. Subsequently, qPCR was run on 1 sample input (I) and the 3 collections from the device (Ff, W, E) using a primer specific to the target oligonucleotide. The capture efficiency of the target-probe complexes was calculated to be about 75% with minimal loss by buffer washing (<1%). The release efficiency by enzymatic cleavage was about 72%, resulting in a total recovery (eluate/input) of 53.6%.

All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications, and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

1-10. (canceled)

11. A method of obtaining a population of target nucleic acid sequences for sequencing comprising:
(a) introducing a pool of oligonucleotide probes to an obtained genomic sample to form target-probe complexes, wherein the pool of oligonucleotide probes comprise reference nucleic acid sequences capable of hybridizing to complementary nucleic acid sequences within the obtained genomic sample, and wherein the oligonucleotide probes comprise a first member of a pair of specific binding entities;
(b) flowing a solution including the formed target-probe complexes through a processing conduit of a microfluidic chip, wherein the processing conduit comprises a chamber including a plurality of beads, and wherein the plurality of beads are functionalized with a second member of the pair of specific binding entities;
(c) flowing at least one fluid through the processing conduit to remove off-target nucleic acids; and
(d) flowing at least one reagent through the processing conduit to obtain the target nucleic acid sequences.

12. The method of claim 11, wherein the at least one reagent is a buffer, and wherein the processing conduit is heated to a temperature ranging from between about 90° C. to about 100° C.

13. The method of claim 11 or 12, wherein the flowing of the at least one fluid is sequentially repeated at least twice or at least three times.

14. The method of any one of claims 11-13, further comprising sequencing the population of target nucleic acid sequences.

15. The method of any one of claims 11-14, wherein the obtained genomic sample comprises cell-free nucleic acids.
